(19) United States
(12) Patent Application Publication
(10) Pub. No.: US 2024/0245717 A1
(43) Pub. Date: Jul. 25, 2024

(54) ANTIVIRAL COMPOUNDS, COMPOSITIONS, AND USES THEREOF

(71) Applicant: Lakewood Amedex, Inc., University Park, FL (US)

(72) Inventors: Kelvin Cooper, Sarasota, FL (US); Steven Parkinson, University Park, FL (US)

(21) Appl. No.: 18/540,055

(22) Filed: Dec. 14, 2023

Related U.S. Application Data
(60) Provisional application No. 63/432,576, filed on Dec. 14, 2022.

(51) Int. Cl. A61K 31/7076; A61K 31/6615; A61K 31/7032; A61K 31/7072; A61P 31/14

(52) U.S. Cl. CPC A61K 31/7076; A61K 31/6615; A61K 31/7032; A61K 31/7072; A61P 31/14

(57) ABSTRACT

Antiviral compounds and compositions of Formula (I) and methods of use are disclosed.

### CROSS-REFERENCE TO RELATED APPLICATION

This application claims priority to U.S. Provisional Patent Application Ser. No. 63/432,576 filed on Dec. 14, 2022, which is incorporated by reference herein in its entirety.

### SEQUENCE LISTING

This application incorporates by reference the material in the XML file titled Sequence_Listing.xml, which was created on Jan. 30, 2024 and is 3.6 KB.

### FIELD

The present disclosure provides novel compositions containing antiviral compounds and uses thereof.

### BACKGROUND

Bisphosphocin® compounds have therapeutic activity. U.S. Pat. No. 7,868,162 discloses Bisphosphocin® compounds.

### SUMMARY

The present disclosure provides a method for treating a coronavirus infection in a patient in need thereof. The method includes administering an effective amount of a composition comprising a compound having the formula.

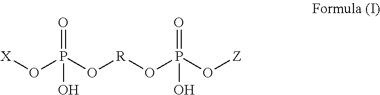

wherein Z and X are independently selected from alkyl, alkyloxyalkyl, alkylaryl, aryl, alkenyl, arylalkyl, arylalkenyl, or alkylarylalkenyl, and each substituent, Z or X, is optionally substituted with other functional groups; and R is selected from alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and each substituent, R, is optionally substituted with other functional groups; or a pharmaceutically acceptable salt thereof.

In some embodiments, the coronavirus is selected from the group consisting of an alphacoronavirus, a betacoronavirus, a gammacoronavirus, a deltacoronavirus, and an omicroncoronavirus, or combinations thereof.

In some embodiments, the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.

In some embodiments, the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, and bat Hp-betacoronavirus Zhejiang2013, or combinations thereof.

In some embodiments, the coronavirus is human SARS-CoV-2.

In some embodiments, the coronavirus is human SARS-CoV.

In some embodiments, the compound comprises a compound having the formula:

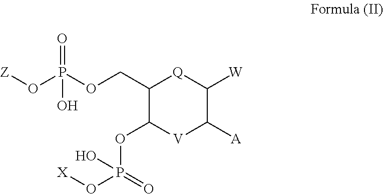

wherein Z and X are independently selected from alkyl, alkylaryl, aryl, alkenyl, arylalkyl, arylalkenyl, or alkylarylalkenyl, and each substituent, Z or X, is optionally substituted with other functional groups; Q is O or S; V is a bond, O, or S; A is H, alkyl, or alkyloxy, and A is optionally substituted with other functional groups; and W is a monocyclic or bicyclic heterocycle; or a pharmaceutically acceptable salt thereof. In some embodiments, the bond to W is to a heteroatom of the heterocycle. In some embodiments, the bond to W is to a carbon atom of the heterocycle.

In some embodiments, the compound comprises a compound having the formula:

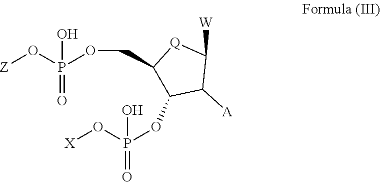

wherein Z and X are independently selected from alkyl, alkylaryl, aryl, alkenyl, arylalkyl, arylalkenyl, or alkylarylalkenyl, and each substituent, Z or X, is optionally substituted with other functional groups; Q is O or S; A is H, alkyl, or alkyloxy, and A is optionally substituted with other functional groups; and W is a monocyclic or bicyclic heterocycle; or a pharmaceutically acceptable salt thereof. In some embodiments, the bond to W is to a heteroatom of the heterocycle.

In some embodiments, the bond to W is to a carbon atom of the heterocycle.

In some embodiments, Z and X are independently alkyl and are substituted with at least one hydroxyl group. In some embodiments, Z and X are both 4-hydroxybutyl. In some embodiments, Z and X are both n-butyl.

In some embodiments, the compound is selected from the group consisting of a compound having the formula:

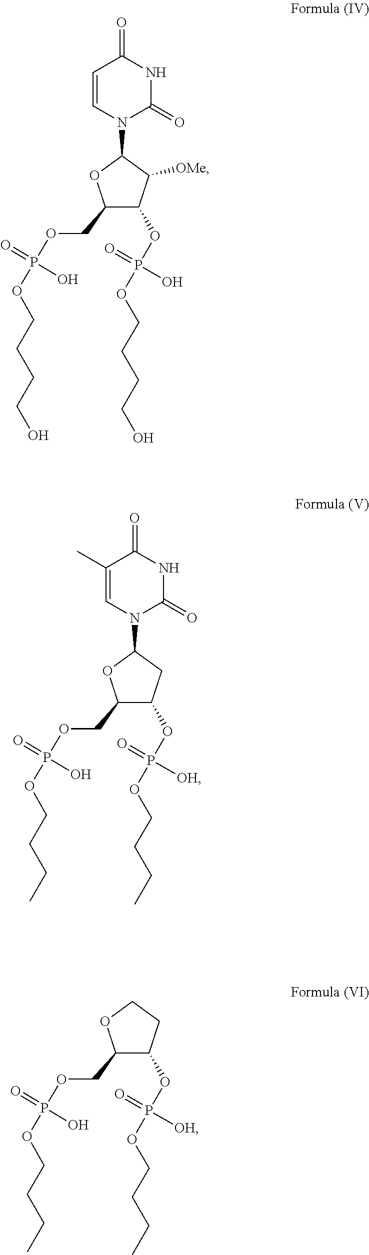 [SMILES: CO[C@@H]1[C@H](OP(=O)(O)OCCCCO)[C@@H](COP(=O)(O)OCCCCO)O[C@H]1n1ccc(=O)[nH]c1=O ; CCCCOP(=O)(O)OC[C@H]1O[C@@H](n2cc(C)c(=O)[nH]c2=O)C[C@@H]1OP(=O)(O)OCCCC ; CCCCOP(=O)(O)OC[C@H]1OCC[C@@H]1OP(=O)(O)OCCCC]

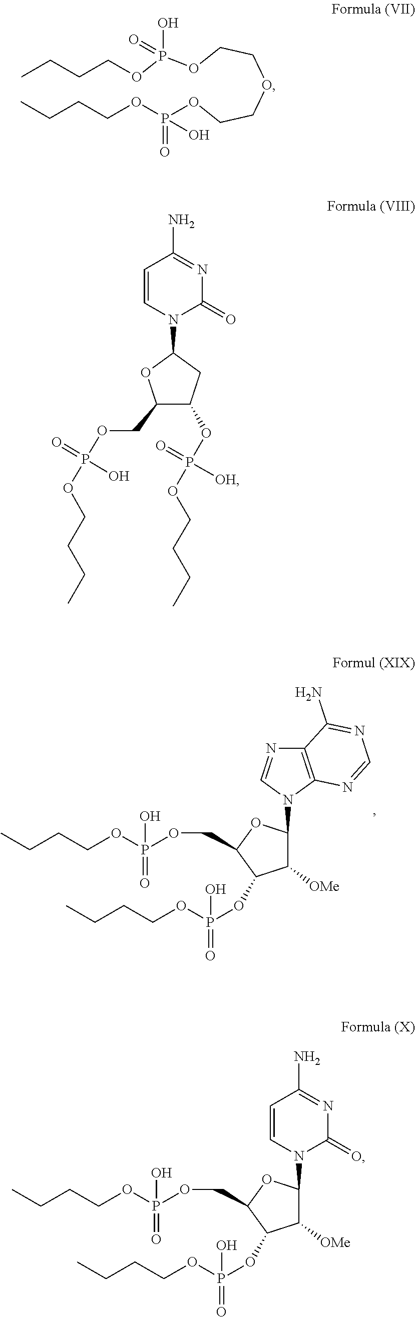 [SMILES: CCCCOP(=O)(O)OCCOCCOP(=O)(O)OCCCC ; CCCCOP(=O)(O)OC[C@H]1O[C@@H](n2ccc(N)nc2=O)C[C@@H]1OP(=O)(O)OCCCC ; CCCCOP(=O)(O)OC[C@H]1O[C@@H](n2cnc3c(N)ncnc32)[C@H](OC)[C@@H]1OP(=O)(O)OCCCC ; CCCCOP(=O)(O)OC[C@H]1O[C@@H](n2ccc(N)nc2=O)[C@H](OC)[C@@H]1OP(=O)(O)OCCCC]

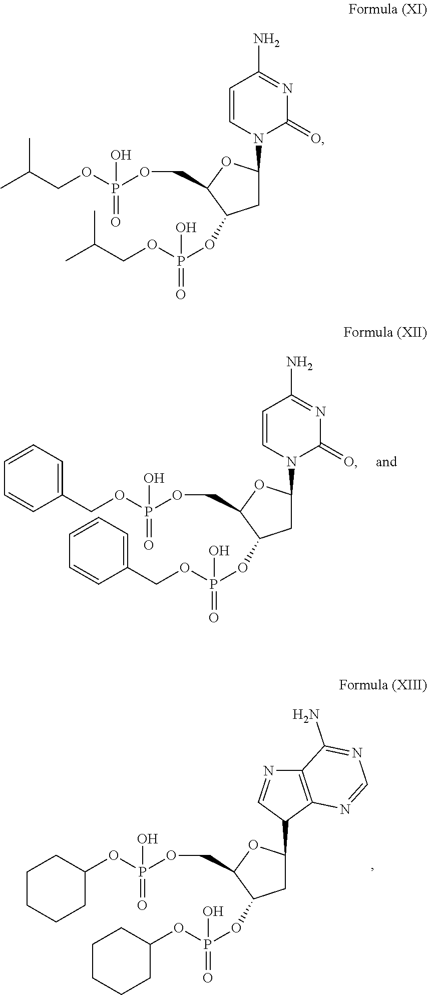 [SMILES: CC(C)COP(=O)(O)OC[C@H]1O[C@@H](n2ccc(N)nc2=O)C[C@@H]1OP(=O)(O)OCC(C)C ; Nc1ccn([C@H]2C[C@H](OP(=O)(O)OCc3ccccc3)[C@@H](COP(=O)(O)OCc3ccccc3)O2)c(=O)n1 ; "and" ; Nc1ncnc2c1N=CC2[C@H]1C[C@H](OP(=O)(O)OC2CCCCC2)[C@@H](COP(=O)(O)OC2CCCCC2)O1]

or combinations thereof; or a pharmaceutically acceptable salt thereof.

In some embodiments, the compound comprises a compound having the formula:

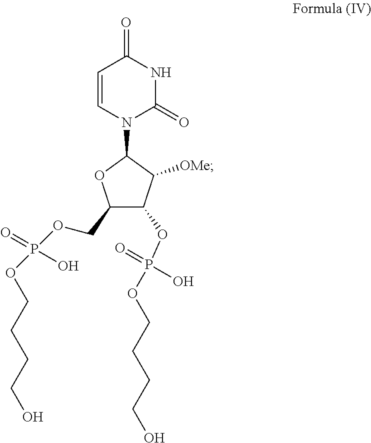 [SMILES: CO[C@@H]1[C@H](OP(=O)(O)OCCCCO)[C@@H](COP(=O)(O)OCCCCO)O[C@H]1n1ccc(=O)[nH]c1=O]

or a pharmaceutically acceptable salt thereof.

In some embodiments, the compound comprises a compound having the formula:

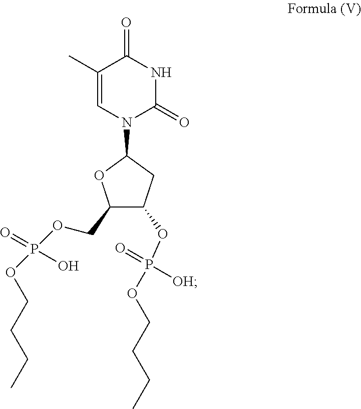 [SMILES: CCCCOP(=O)(O)OC[C@H]1O[C@@H](n2cc(C)c(=O)[nH]c2=O)C[C@@H]1OP(=O)(O)OCCCC]

or a pharmaceutically acceptable salt thereof.

In some embodiments, the compound comprises a compound having the formula:

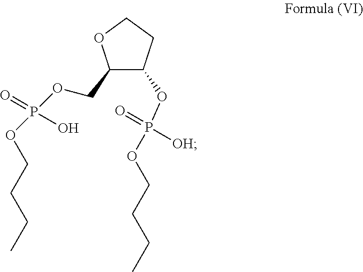 [SMILES: CCCCOP(=O)(O)OC[C@H]1OCC[C@@H]1OP(=O)(O)OCCCC]

or a pharmaceutically acceptable salt thereof.

In some embodiments, the compound comprises a compound having the formula:

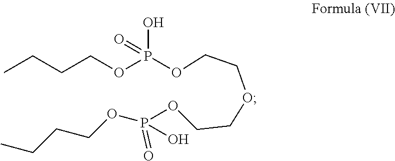 [SMILES: CCCCOP(=O)(O)OCCOCCOP(=O)(O)OCCCC]

or a pharmaceutically acceptable salt thereof.

In some embodiments, the compound comprises a compound having the formula:

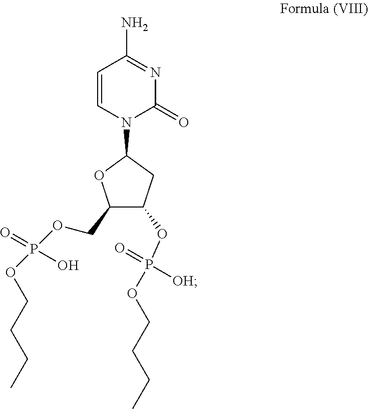 [SMILES: CCCCOP(=O)(O)OC[C@H]1O[C@@H](n2ccc(N)nc2=O)C[C@@H]1OP(=O)(O)OCCCC]

or a pharmaceutically acceptable salt thereof.

In some embodiments, the compound comprises a compound having the formula:

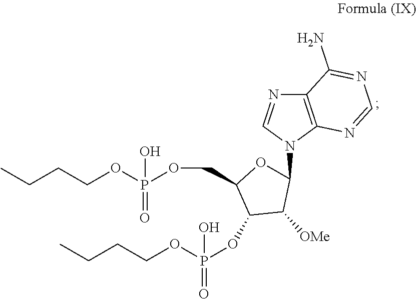 [SMILES: CCCCOP(=O)(O)OC[C@H]1O[C@@H](n2cnc3c(N)ncnc32)[C@H](OC)[C@@H]1OP(=O)(O)OCCCC]

or a pharmaceutically acceptable salt thereof.

In some embodiments, the compound comprises a compound having the formula:

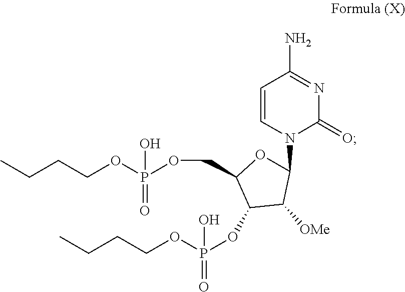 [SMILES: CCCCOP(=O)(O)OC[C@H]1O[C@@H](n2ccc(N)nc2=O)[C@H](OC)[C@@H]1OP(=O)(O)OCCCC]

or a pharmaceutically acceptable salt thereof.

In some embodiments, the compound comprises a compound having the formula:

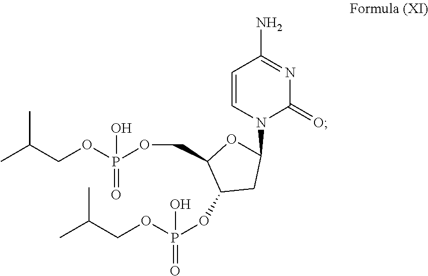 [SMILES: CC(C)COP(=O)(O)OC[C@H]1O[C@@H](n2ccc(N)nc2=O)C[C@@H]1OP(=O)(O)OCC(C)C]

or a pharmaceutically acceptable salt thereof.

In some embodiments, the compound comprises a compound having the formula:

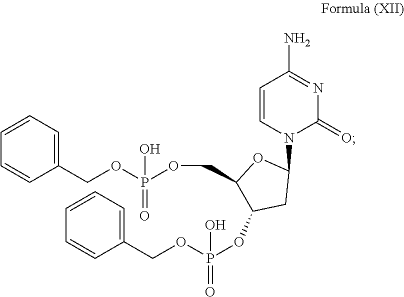 [SMILES: Nc1ccn([C@H]2C[C@H](OP(=O)(O)OCc3ccccc3)[C@@H](COP(=O)(O)OCc3ccccc3)O2)c(=O)n1]

or a pharmaceutically acceptable salt thereof.

In some embodiments, the compound comprises a compound having the formula:

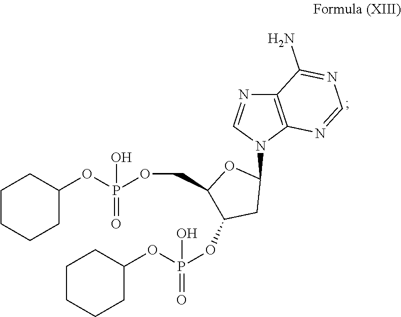 [SMILES: Nc1ncnc2c1ncn2[C@H]1C[C@H](OP(=O)(O)OC2CCCCC2)[C@@H](COP(=O)(O)OC2CCCCC2)O1]

or a pharmaceutically acceptable salt thereof.

In some embodiments, the pharmaceutically acceptable salt is selected from the group consisting of an ammonium salt, a calcium salt, a sodium salt, a potassium salt, a magnesium salt, and a cobalt salt, or combinations thereof.

In some embodiments, the compound or the pharmaceutically acceptable salt thereof, is a protonated compound having a pH when dissolved in water of about pH 3 to about pH 5.

In some embodiments, the compound or the pharmaceutically acceptable salt thereof, is a protonated compound having a pH when dissolved in water of about pH 3 to about pH 4.

In some embodiments, the composition further comprises one or more pharmaceutically acceptable carriers, excipients, or diluents.

In some embodiments, the composition further comprises at least one additional active ingredient.

In some embodiments, the patient is a mammal. In some embodiments, the patient is a human.

In some embodiments, the administering is oral administration. In some embodiments, the oral administration comprises administering the composition as a capsule, a tablet, a powder, a liquid suspension, a solution, an elixir, a syrup, or a concentrated drop.

In some embodiments, the administering is inhalation administration. In some embodiments, the inhalation administration comprises administering the composition as a dry powder, a solution, a suspension, or an aerosol.

In some embodiments, the administering is topical administration. In some embodiments, the topical administration comprises administering the composition as a lotion, a gel, a cream, an ointment, an oil, a solution, a suspension, or an emulsion.

In some embodiments, the administering is intravesical administration.

In some embodiments, the administering is administration via a catheter.

In some embodiments, the administering is parenteral administration. In some embodiments, the administering is administration via a needle.

In some embodiments, the administering comprises administering as a gel comprising a polyhydric alcohol.

In some embodiments, the administering is carried out as a multiple dose regimen. In some embodiments, the multiple dose regimen is a time period of up to about one month. In some embodiments, the multiple dose regimen is a time period of up to about two months. In some embodiments, the multiple dose regimen is a time period of up to about three months. In some embodiments, the multiple dose regimen is a time period of up to about four months.

In some embodiments, the administering is carried out one time per day. In some embodiments, the administering is carried out two times per day. In some embodiments, the administering is carried out three times per day. In some embodiments, the administering is carried out four times per day.

In some embodiments, the administering is carried out for at least 1 day. In some embodiments, the administering is carried out for at least 7 days. In some embodiments, the administering is carried out for at least 14 days. In some embodiments, the administering is carried out for at least 21 days.

In some embodiments, the other functional groups are selected from the group consisting of an amino, a carboxyl, a hydroxy, an alkoxy, a ketone, an aldehyde, and a halogen, or combinations thereof.

Additional aspects and embodiments will be apparent from the Detailed Description and from the claims.

### BRIEF DESCRIPTION OF THE DRAWINGS

The teachings of some embodiments of the present disclosure will be better understood by reference to the description taken in conjunction with the accompanying drawings, wherein:

FIG. 1 shows body weight measurements for a set of hamsters infected with 5×10⁴ plaque-forming units (hereinafter “PFU”) SARS-CoV-2 and a set of hamsters infected with 5×10³ PFU SARS-CoV-2;

FIG. 2A shows body weight measurements of uninfected, untreated control hamsters up to 7 days after the start of the study;

FIG. 2B shows body weight measurements of the infected, untreated control hamsters that received only the drug vehicle according to embodiments;

FIG. 2C provides body weight measurements of a population of hamsters treated with Nu-8 (compound of Formula (VIII)) and a population of hamsters treated with Nu-3 (compound of Formula (V)) according to embodiments;

FIG. 3 provides the viral titers from lungs harvested from hamsters according to embodiments;

FIG. 4 provides the results of a neutralization assay for Nu-10 (compound of Formula (X)) according to embodiments; and

FIG. 5 provides the results of a neutralization assay for Nu-11 (compound of Formula (IX)) according to embodiments.

### DETAILED DESCRIPTION

The embodiments described below are not intended to be exhaustive or to limit the invention to the precise forms disclosed in the following detailed description. Rather, the embodiments are chosen and described so that others skilled in the art may appreciate and understand the principles and practices of this disclosure.

The present disclosure provides a method for treating a viral infection in a patient in need thereof. The method includes administering an effective amount of a composition comprising at least one compound of Formula (I) or a pharmaceutically acceptable salt thereof:

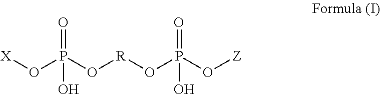

where Z and X are independently selected from alkyl, alkyloxyalkyl, alkylaryl, aryl, alkenyl, arylalkyl, arylalkenyl, or alkylarylalkenyl, and each substituent, Z or X, is optionally substituted with other functional groups; and R is selected from alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and each substituent, R, is optionally substituted with other functional groups. In some embodiments, the viral infection is caused by a coronavirus.

As used herein, the term “other functional groups” refers to one or more atoms with distinctive chemical properties, the reactivity of which may be modified by other functional groups present. Functional groups include, but are not limited to, alkyl, alkenyl, alkynyl, phenyl, haloalkyl (e.g., fluoroalkyl, chloroalkyl, bromoalkyl, iodoalkyl), hydroxyl, carbonyl, aldehyde, haloformyl, carbonate ester, carboxylate, carboxyl, carboalkoxy, hydroperoxy, peroxy, ether, hemiacetal, hemiketal, acetal, ketal, orthoester, methylenedioxy, orthocarbonate ester, carboxylic anhydride, carboxamide, amidine, amine, imine, imide, azide, diimide, cyanate, isocyante, nitrate, nitrile, isonitrile, nitrosooxy, nitro, nitroso, oxime, pyridyl, carbamate, sulfhydryl, sulfide, disulfide, sulfinyl, sulfonyl, sulfino, sulfo, thiocyanate, isothiocyanate, carbonothioyl, thiocarboxylic acid (e.g., carbothioic S-acid and carbothioic O-acid), thiolester, thionoester, carbodithioic acid, carbodithio, phosphino, phosphono, phosphate, borono, boronate, borino, borinate, alkyllithium, alkylmagnesium halide, alkylaluminum, and silyl ether.

In some embodiments, the compound of Formula (I) may include or be a compound of Formula (II) or a pharmaceutically acceptable salt thereof:

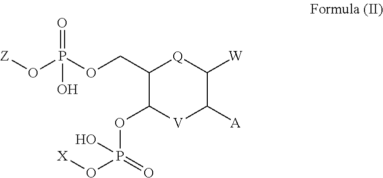

where Z and X are independently selected from alkyl, alkylaryl, aryl, alkenyl, arylalkyl, arylalkenyl, or alkylarylalkenyl, and each substituent, Z or X, is optionally substituted with other functional groups; Q is O or S; A is H, alkyl, or alkyloxy, and A is optionally substituted with other functional groups; and W is a monocyclic or bicyclic heterocycle. In some embodiments, the bond to W is to a heteroatom of the heterocycle. In some embodiments, the bond to W is to a carbon atom of the heterocycle.

In some embodiments, the compound of Formula (I) may include or be a compound of Formula (III) or a pharmaceutically acceptable salt thereof:

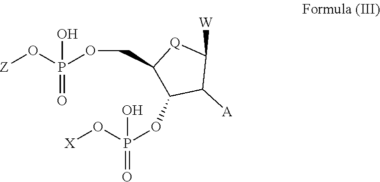

where Z and X are independently selected from alkyl, alkylaryl, aryl, alkenyl, arylalkyl, arylalkenyl, or alkylarylalkenyl, and each substituent, Z or X, is optionally substituted with other functional groups; Q is O or S; V is a bond, O, or S; A is H, alkyl, or alkyloxy, and A is optionally substituted with other functional groups; and W is a monocyclic or bicyclic heterocycle. In some embodiments, the bond to W is to a heteroatom of the heterocycle. In some embodiments, the bond to W is to a carbon atom of the heterocycle. In some embodiments, Z and X are independently alkyl and are substituted with at least one hydroxyl group. In some embodiments, Z and X are both 4-hydroxybutyl. In some embodiments, Z and X are both n-butyl.

In some embodiments, the compound of Formula (I) may include or be a compound of Formula (IV), a compound of Formula (V), a compound of Formula (VI), a compound of Formula (VII), a compound of Formula (VIII), a compound of Formula (IX), a compound of Formula (X), a compound of Formula (XI), a compound of Formula (XII), and a compound of Formula (XIII), or combinations thereof:

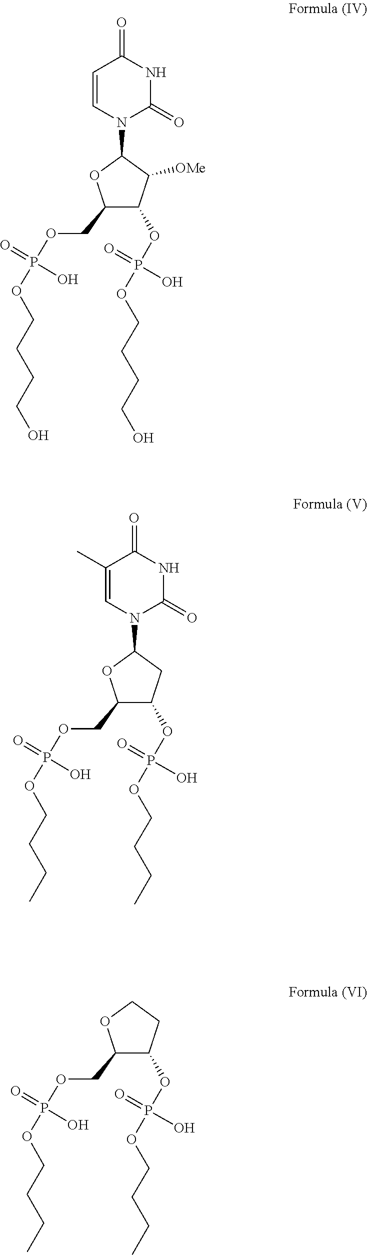 [SMILES: CO[C@@H]1[C@H](OP(=O)(O)OCCCCO)[C@@H](COP(=O)(O)OCCCCO)O[C@H]1n1ccc(=O)[nH]c1=O ; CCCCOP(=O)(O)OC[C@H]1O[C@@H](n2cc(C)c(=O)[nH]c2=O)C[C@@H]1OP(=O)(O)OCCCC ; CCCCOP(=O)(O)OC[C@H]1OCC[C@@H]1OP(=O)(O)OCCCC]

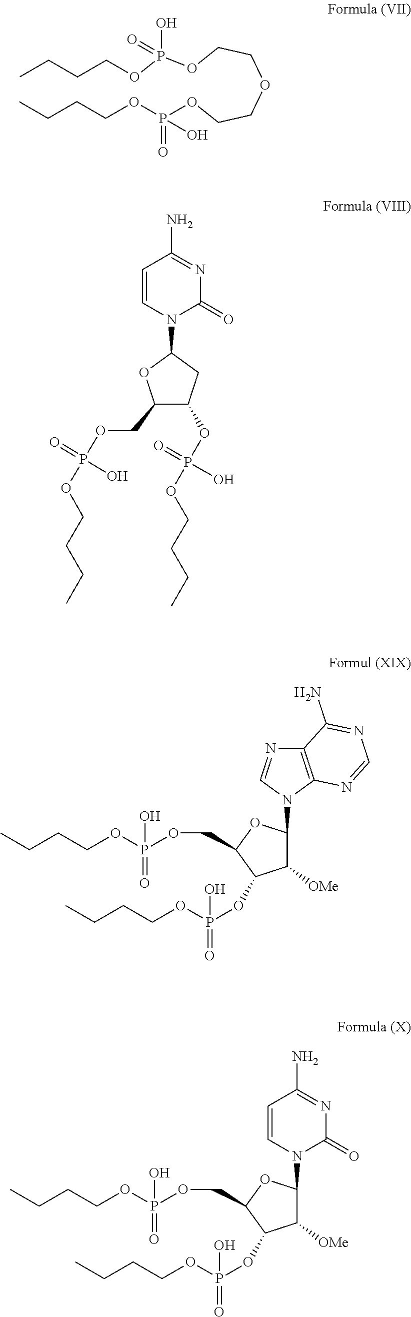 [SMILES: CCCCOP(=O)(O)OCCOCCOP(=O)(O)OCCCC ; CCCCOP(=O)(O)OC[C@H]1O[C@@H](n2ccc(N)nc2=O)C[C@@H]1OP(=O)(O)OCCCC ; CCCCOP(=O)(O)OC[C@H]1O[C@@H](n2cnc3c(N)ncnc32)[C@H](OC)[C@@H]1OP(=O)(O)OCCCC ; CCCCOP(=O)(O)OC[C@H]1O[C@@H](n2ccc(N)nc2=O)[C@H](OC)[C@@H]1OP(=O)(O)OCCCC]

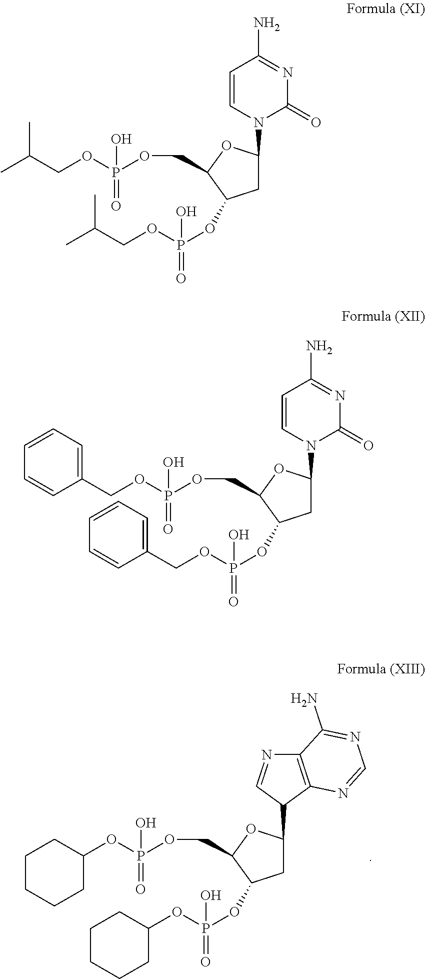 [SMILES: CC(C)COP(=O)(O)OC[C@H]1O[C@@H](n2ccc(N)nc2=O)C[C@@H]1OP(=O)(O)OCC(C)C ; Nc1ccn([C@H]2C[C@H](OP(=O)(O)OCc3ccccc3)[C@@H](COP(=O)(O)OCc3ccccc3)O2)c(=O)n1 ; Nc1ncnc2c1N=CC2[C@H]1C[C@H](OP(=O)(O)OC2CCCCC2)[C@@H](COP(=O)(O)OC2CCCCC2)O1]

The chemical name of the compound of Formula (IV) is (4-hydroxybutyl)-phosphate-5′-uridine-2′-methoxy-3′-phosphate-(4-hydroxybutyl). The molecular formula of the compound of Formula (IV) is C₁₈H₃₂N₂O₁₄P₂. The molecular weight of the compound of Formula (IV) is 562.4 Da. The compound of Formula (IV) is also referred to herein as Nu-2, which such terms are used interchangeably herein. In some embodiments, a compound of Formula (IV) includes a ribose, two phosphate groups, two hydroxybutyl groups, and a uracil.

The chemical name of the compound of Formula (V) is butyl-phosphate-5′-thymidine-3′-phosphate-butyl. The molecular formula of the compound of Formula (V) is C₁₈H₃₂N₂O₁₁P₂. The molecular weight of the compound of Formula (V) is 514.4 Da. The compound of Formula (V) is also referred to herein as Nu-3, which such terms are used interchangeably herein. In some embodiments, a compound of Formula (V) includes a ribose, two phosphate groups, two butyl groups, and a thymine.

The chemical name of the compound of Formula (VI) is butyl-phosphate-5′-ribose-3′-phosphate-butyl. The molecular formula of the compound of Formula (VI) is C₁₃H₂₈O₉P₂. The molecular weight of the compound of Formula (VI) is 390.3 Da. The compound of Formula (VI) is also referred to herein as Nu-4, which such terms are used interchangeably herein. In some embodiments, a compound of Formula (VI) includes a ribose, two phosphate groups, and two butyl groups.

The chemical name of the compound of Formula (VII) is P,P′-(oxydi-2,1-ethanediyl) bis(P-butyl phosphate). The molecular formula of the compound of Formula (VII) is C₁₂H₂₆O₉P₂. The molecular weight of the compound of Formula (VII) is 376.28 Da. The compound of Formula (VII) is also referred to herein as Nu-5, which such terms are used interchangeably herein. In some embodiments, a compound of Formula (VII) includes two phosphate groups and two butyl groups.

The chemical name of the compound of Formula (VIII) is ((2R,3S,5R)-5-(4-amino-2-oxopyrimidin-1(2H)-yl)-3-((butoxyoxidophosphor-yl)oxy)tetrahydrofuran-2-yl)methyl butyl phosphate. The molecular formula of the compound of Formula (VIII) is C₁₇H₃₁N₃O₁₀P₂. The molecular weight of the compound of Formula (VIII) is 499.38 Da. The compound of Formula (VIII) is also referred to herein as Nu-8, which such terms are used interchangeably herein. In some embodiments, a compound of the present disclosure includes a ribose, two phosphate groups, two butyl groups, and a cytosine.

The chemical name of the compound of Formula (IX) is (2R,3R,4R,5R)-5-(6-aminopurin-9-yl)-3-((butoxy(hydroxy)phosphoryl)oxy)-4-methoxytetrahydrofuran-2-yl)methyl butyl hydrogen phosphate. The molecular formula of the compound of Formula (IX) is C₁₉H₃₃N₅O₁₀P₂. The molecular weight of the compound of Formula (IX) is 553.44 Da. The compound of Formula (IX) is also referred to herein as Nu-11, Compound (IX) or Cmpd 1398 which such terms are used interchangeably herein. In some embodiments, Nu-11 includes a ribose, two phosphate groups, two butyl groups, and a guanine.

The chemical name of the compound of Formula (X) is ((2R,3R,4R,5R)-5-(4-amino-2-oxopyrimidin-1(2H)-yl)-3-((butoxy(hydroxy)phosphoryl)oxy)-4-methoxytetrahydrofuran-2-yl) methyl butyl hydrogen phosphate. The molecular formula of the compound of Formula (X) is C₁₈H₃₃N₃O₁₁P₂. The molecular weight of the compound of Formula (X) is 529.42 Da. The compound of Formula (X) is also referred to herein as Nu-10, Compound (X) or Cmpd 1261, which such terms are used interchangeably herein. In some embodiments, Nu-10 includes a ribose, two phosphate groups, two butyl groups, and a cytosine.

The chemical name of the compound of Formula (XI) is ((2R,3S,5R)-5-(4-amino-2-oxopyrimidin-1(2H)-yl)-3-((hydroxy(isobutoxy)phosphoryl)oxy)tetrahydrofuran-2-yl)methyl isobutyl hydrogen phosphate. The molecular formula of the compound of Formula (XI) is C₁₇H₃₁N₃O₁₀P₂. The molecular weight of the compound of Formula (XI) is 499.39 Da. The compound of Formula (XI) is also referred to herein as Nu-13, Compound (XI) or Cmpd 211, which such terms are used interchangeably herein. In some embodiments, Nu-13 includes a ribose, two phosphate groups, two isobutyl groups, and a cytosine.

The chemical name of the compound of Formula (XII) is ((2R,3S,5R)-5-(4-amino-2-oxopyrimidin-1(2H)-yl)-3-(((benzyloxy)(hydroxy)phosphoryl)oxy)tetrahydrofuran-2-yl)methyl benzyl hydrogen phosphate. The molecular formula of the compound of Formula (XII) is C₂₃H₂₇N₃O₁₀P₂. The molecular weight of the compound of Formula (XII) is 567.42 Da. The compound of Formula (XII) is also referred to herein as Nu-14, Compound (XII) or Cmpd 841, which such terms are used interchangeably herein. In some embodiments, Nu-14 includes a ribose, two phosphate groups, two benzyl groups, and a cytosine.

The chemical name of the compound of Formula (XIII) is (2R,3S,5R)-5-(6-aminopurin-9-yl)-3-((cyclohexyloxy(hydroxy)phosphoryl)oxy)tetrahydrofuran-2-yl)methyl cyclohexyl hydrogen phosphate. The molecular formula of the compound of Formula (XIII) is C₂₂H₃₅N₅O₉P₂. The molecular weight of the compound of Formula (XIII) is 575.49 Da. The compound of Formula (XIII) is also referred to herein as Nu-12, Compound (XIII) or Cmpd 558, which such terms are used interchangeably herein. In some embodiments, Nu-12 includes a ribose, two phosphate groups, two cyclohexyl groups, and a guanine.

The compound of Formula (I) is described with reference to the specific compound illustrated herein. In addition, the compound of Formula (I) may exist in any number of different forms or derivatives, all within the scope of the present disclosure. Alternative forms or derivatives include, for example, pharmaceutically acceptable salts, prodrugs and active metabolites, tautomers, and solid forms, including without limitation different crystal forms, polymorphic or amorphous solids, including hydrates and solvates thereof, and other forms.

Unless specified to the contrary, specification of the compound of Formula (I) herein includes pharmaceutically acceptable salts of such compound. Thus, the compound of Formula (I) can be in the form of pharmaceutically acceptable salts or can be formulated as pharmaceutically acceptable salts. Contemplated pharmaceutically acceptable salt forms of the present disclosure include, without limitation, mono, bis, tris, tetrakis, and so on. Pharmaceutically acceptable salts of the present disclosure are non-toxic in the amounts and concentrations at which such pharmaceutically acceptable salts are administered. The preparation of such pharmaceutically acceptable salts of the present disclosure can facilitate the pharmacological use by altering the physical characteristics of a compound of the present disclosure without preventing it from exerting its physiological effect.

As used herein, the term “pharmaceutically acceptable,” with respect to salts and formulation components such as carriers, excipients, and diluents, refers to those salts and components which are not deleterious to a patient and which are compatible with other ingredients, active ingredients, salts or components. Pharmaceutically acceptable includes “veterinarily acceptable,” and thus includes both human and non-human mammal applications independently.

As used herein, the term “pharmaceutically acceptable salt” refers to salts commonly used to form alkali metal salts and to form addition salts of free acids or free bases. Such salts include, for example, the physiologically acceptable salts listed in Handbook of Pharmaceutical Salts: Properties, Selection and Use, P. H. Stahl and C. G. Wermuth (Eds.), Wiley-VCH, New York, 2002, which are known to the skilled artisan. Salt formation can occur at one or more positions having labile protons. The pharmaceutically acceptable salts of a compound of the present disclosure include both acid addition salts and base addition salts.

In some embodiments, suitable pharmaceutically acceptable acid addition salts of the compounds of the present disclosure may be prepared from an inorganic acid or an organic acid. Examples of such inorganic acids include without limitation hydrochloric, hydrobromic, hydroiodic, nitric, carbonic, sulfuric, and phosphoric acid. Appropriate organic acids include without limitation aliphatic, cycloaliphatic, aromatic, arylaliphatic, heterocyclic, carboxylic, and sulfonic classes of organic acids, examples of which are formic, acetic, propionic, succinic, glycolic, gluconic, maleic, embonic (pamoic), methanesulfonic, ethanesulfonic, 2-hydroxyethanesulfonic, pantothenic, benzenesulfonic, toluenesulfonic, sulfanilic, mesylic, cyclohexylaminosulfonic, stearic, algenic, beta-hydroxybutyric, malonic, galactic, and galacturonic acid, to name a few. Pharmaceutically acceptable acidic/anionic salts also include, the acetate, benzenesulfonate, benzoate, bicarbonate, bitartrate, bromide, calcium edetate, camsylate, carbonate, chloride, citrate, dihydrochloride, edetate, edisylate, estolate, esylate, fumarate, glyceptate, gluconate, glutamate, glycollylarsanilate, hexylresorcinate, hydrobromide, hydrochloride, hydroxynaphthoate, iodide, isethionate, lactate, lactobionate, malate, maleate, malonate, mandelate, mesylate, methylsulfate, mucate, napsylate, nitrate, pamoate, pantothenate, phosphate/diphospate, polygalacturonate, salicylate, stearate, subacetate, succinate, sulfate, hydrogensulfate, tannate, tartrate, teoclate, tosylate, and triethiodide salts, among others.

In some embodiments, suitable pharmaceutically acceptable base addition salts of the compounds of the present disclosure include without limitation metallic salts made from aluminum, calcium, lithium, magnesium, potassium, sodium and zinc, or organic salts made from N,N′-dibenzylethylene-diamine, chloroprocaine, choline, diethanolamine, ethylenediamine, N-methylglucamine, lysine, arginine, and procaine. All of these salts may be prepared by conventional means from a compound of the present disclosure by treating a compound of the present disclosure with the appropriate acid or base. Pharmaceutically acceptable basic/cationic salts also include diethanolamine, ammonium, ethanolamine, piperazine and triethanolamine salts, to name a few. In some embodiments, a pharmaceutically acceptable salt of the present disclosure comprises a monovalent cation or a divalent cation.

In some embodiments, a pharmaceutically acceptable salt of the present disclosure is selected from the group consisting of an ammonium salt, a calcium salt, a sodium salt, a potassium salt, a magnesium salt, and a cobalt salt.

In some embodiments, the ammonium salt is diammonium (4-hydroxybutyl)-phosphate-5′-uridine-2′-methoxy-3′-phosphate-(4-hydroxybutyl). In some embodiments, the calcium salt is calcium (4-hydroxybutyl)-phosphate-5′-uridine-2′-methoxy-3′-phosphate-(4-hydroxybutyl). In some embodiments, the sodium salt is disodium (4-hydroxybutyl)-phosphate-5′-uridine-2′-methoxy-3′-phosphate-(4-hydroxybutyl). In some embodiments, the potassium salt is dipotassium (4-hydroxybutyl)-phosphate-5′-uridine-2′-methoxy-3′-phosphate-(4-hydroxybutyl). In some embodiments, the magnesium salt is magnesium (4-hydroxybutyl)-phosphate-5′-uridine-2′-methoxy-3′-phosphate-(4-hydroxybutyl). In some embodiments, the cobalt salt is cobalt (4-hydroxybutyl)-phosphate-5′-uridine-2′-methoxy-3′-phosphate-(4-hydroxybutyl).

In some embodiments, the ammonium salt is diammonium butyl-phosphate-5′-thymidine-3′-phosphate-butyl. In some embodiments, the calcium salt is calcium butyl-phosphate-5′-thymidine-3′-phosphate-butyl. In some embodiments, the sodium salt is disodium butyl-phosphate-5′-thymidine-3′-phosphate-butyl. In some embodiments, the potassium salt is dipotassium butyl-phosphate-5′-thymidine-3′-phosphate-butyl. In some embodiments, the magnesium salt is magnesium butyl-phosphate-5′-thymidine-3′-phosphate-butyl. In some embodiments, the cobalt salt is cobalt butyl-phosphate-5′-thymidine-3′-phosphate-butyl. In some embodiments, the ammonium salt is diammonium butyl-phosphate-5′-ribose-3′-phosphate-butyl. In some embodiments, the calcium salt is calcium butyl-phosphate-5′-ribose-3′-phosphate-butyl. In some embodiments, the sodium salt is disodium butyl-phosphate-5′-ribose-3′-phosphate-butyl. In some embodiments, the potassium salt is dipotassium butyl-phosphate-5′-ribose-3′-phosphate-butyl. In some embodiments, the magnesium salt is magnesium butyl-phosphate-5′-ribose-3′-phosphate-butyl. In some embodiments, the cobalt salt is cobalt butyl-phosphate-5′-ribose-3′-phosphate-butyl.

In some embodiments, the ammonium salt is diammonium oxydi(ethane-2,1-diyl) dibutyl bisphosphate. In some embodiments, the calcium salt is calcium oxydi(ethane-2,1-diyl) dibutyl bisphosphate. In some embodiments, the sodium salt is disodium oxydi(ethane-2,1-diyl) dibutyl bisphosphate. In some embodiments, the potassium salt is dipotassium oxydi(ethane-2,1-diyl) dibutyl bisphosphate. In some embodiments, the magnesium salt is magnesium oxydi(ethane-2,1-diyl) dibutyl bisphosphate. In some embodiments, the cobalt salt is cobalt oxydi(ethane-2,1-diyl) dibutyl bisphosphate.

In some embodiments, the ammonium salt is ammonium ((2R,3S,5R)-5-(4-amino-2-oxopyrimidin-1(2H)-yl)-3-((butoxyoxidophosphor-yl)oxy)tetrahydrofuran-2-yl)methyl butyl phosphate. In some embodiments, the calcium salt is calcium ((2R,3S,5R)-5-(4-amino-2-oxopyrimidin-1(2H)-yl)-3-((butoxyoxidophosphor-yl)oxy)tetrahydrofuran-2-yl)methyl butyl phosphate. In some embodiments, the sodium salt is sodium ((2R,3S,5R)-5-(4-amino-2-oxopyrimidin-1(2H)-yl)-3-((butoxyoxidophosphor-yl)oxy)tetrahydrofuran-2-yl)methyl butyl phosphate. In some embodiments, the potassium salt is potassium ((2R,3S,5R)-5-(4-amino-2-oxopyrimidin-1(2H)-yl)-3-((butoxyoxidophosphor-yl)oxy)tetrahydrofuran-2-yl)methyl butyl phosphate. In some embodiments, the magnesium salt is magnesium ((2R,3S,5R)-5-(4-amino-2-oxopyrimidin-1(2H)-yl)-3-((butoxyoxidophosphor-yl)oxy)tetrahydrofuran-2-yl)methyl butyl phosphate. In some embodiments, the cobalt salt is cobalt ((2R,3S,5R)-5-(4-amino-2-oxopyrimidin-1(2H)-yl)-3-((butoxyoxidophosphor-yl)oxy)tetrahydrofuran-2-yl)methyl butyl phosphate.

In some embodiments, the ammonium salt is (2R,3R,4R,5R)-5-(6-aminopurin-9-yl)-3-((butoxy(oxy)phosphoryloxy)-4-methoxytetrahydrofuran-2-yl)methyl butyl phosphonate diammonium salt. In some embodiments, the calcium salt is (2R,3R,4R,5R)-5-(6-aminopurin-9-yl)-3-((butoxy(oxy)phosphoryloxy)-4-methoxytetrahydrofuran-2-yl)methyl butyl phosphonate calcium salt. In some embodiments, the sodium salt is(2R,3R,4R,5R)-5-(6-aminopurin-9-yl)-3-((butoxy(oxy)phosphoryloxy)-4-methoxytetrahydrofuran-2-yl)methyl butyl phosphonate disodium salt. In some embodiments, the potassium salt is (2R,3R,4R,5R)-5-(6-aminopurin-9-yl)-3-((butoxy(oxy)phosphoryloxy)-4-methoxytetrahydrofuran-2-yl)methyl butyl phosphonate dipotassium salt. In some embodiments, the magnesium salt is (2R,3R,4R,5R)-5-(6-aminopurin-9-yl)-3-((butoxy(oxy)phosphoryloxy)-4-methoxytetrahydrofuran-2-yl)methyl butyl phosphonate magnesium salt. In some embodiments, the cobalt salt is (2R,3R,4R,5R)-5-(6-aminopurin-9-yl)-3-((butoxy(oxy)phosphoryloxy)-4-methoxytetrahydrofuran-2-yl)methyl butyl phosphonate cobalt salt.

In some embodiments, the ammonium salt is ((2R,3R,4R,5R)-5-(4-amino-2-oxopyrimidin-1(2H)-yl)-3-((butoxy(oxy)phosphoryloxy)-4-methoxytetrahydrofuran-2-yl) methyl butyl phosphonate diammonium salt. In some embodiments, the calcium salt is ((2R,3R,4R,5R)-5-(4-amino-2-oxopyrimidin-1(2H)-yl)-3-((butoxy(oxy)phosphoryloxy)-4-methoxytetrahydrofuran-2-yl) methyl butyl phosphonate calcium salt. In some embodiments, the sodium salt is ((2R,3R,4R,5R)-5-(4-amino-2-oxopyrimidin-1(2H)-yl)-3-((butoxy(oxy)phosphoryloxy)-4-methoxytetrahydro-furan-2-yl) methyl butyl phosphonate disodium salt. In some embodiments, the potassium salt is ((2R,3R,4R,5R)-5-(4-amino-2-oxopyrimidin-1(2H)-yl)-3-((butoxy(oxy)phosphoryloxy)-4-methoxytetrahydrofuran-2-yl) methyl butyl phosphonate dipotassium salt. In some embodiments, the magnesium salt is ((2R,3R,4R,5R)-5-(4-amino-2-oxopyrimidin-1(2H)-yl)-3-((butoxy(oxy)phosphoryloxy)-4-methoxytetrahydrofuran-2-yl) methyl butyl phosphonate magnesium salt. In some embodiments, the cobalt salt is ((2R,3R,4R,5R)-5-(4-amino-2-oxopyrimidin-1(2H)-yl)-3-((butoxy(oxy)phosphoryloxy)-4-methoxytetrahydrofuran-2-yl) methyl butyl phosphonate cobalt salt.

In some embodiments, the ammonium salt is ((2R,3S,5R)-5-(4-amino-2-oxopyrimidin-1(2H)-yl)-3-(((isobutoxy)(oxy)phosphoryloxy)tetrahydrofuran-2-yl)methyl isobutyl phosphonate diammonium salt. In some embodiments, the calcium salt is ((2R,3S,5R)-5-(4-amino-2-oxopyrimidin-1(2H)-yl)-3-(((isobutoxy)(oxy)phosphoryloxy)tetrahydrofuran-2-yl)methyl isobutyl phosphonate calcium salt. In some embodiments, the sodium salt is ((2R,3S,5R)-5-(4-amino-2-oxopyrimidin-1(2H)-yl)-3-(((isobutoxy)(oxy)phosphoryloxy)tetrahydrofuran-2-yl)methyl isobutyl phosphonate disodium salt. In some embodiments, the potassium salt is ((2R,3S,5R)-5-(4-amino-2-oxopyrimidin-1(2H)-yl)-3-(((isobutoxy)(oxy)phosphoryloxy)tetra-hydrofuran-2-yl)methyl isobutyl phosphonate dipotassium salt. In some embodiments, the magnesium salt is ((2R,3S,5R)-5-(4-amino-2-oxopyrimidin-1(2H)-yl)-3-(((isobutoxy)(oxy)phosphoryloxy)tetrahydrofuran-2-yl)methyl isobutyl phosphonate magnesium salt. In some embodiments, the cobalt salt is ((2R,3S,5R)-5-(4-amino-2-oxopyrimidin-1(2H)-yl)-3-(((isobutoxy)(oxy)phosphoryloxy)tetrahydrofuran-2-yl)methyl isobutyl phosphonate cobalt salt.

In some embodiments, the ammonium salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate diammonium salt. In some embodiments, the calcium salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate calcium salt. In some embodiments, the sodium salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate disodium salt. In some embodiments, the potassium salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate dipotassium salt. In some embodiments, the magnesium salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate magnesium salt. In some embodiments, the cobalt salt is ((2R,3S,5R)-5-(4-amino-2-oxopyrimidin-1(2H)-yl)-3-(((benzyloxy)(oxy)phosphoryloxy)tetrahydrofuran-2-yl)methyl benzyl phosphonate cobalt salt.

In some embodiments, the ammonium salt is (2R,3S,5R)-5-(6-aminopurin-9-yl)-3-((cyclohexyloxy(oxy)phosphoryloxy)tetrahydrofuran-2-yl)methyl cyclohexyl phosphonate diammonium salt. In some embodiments, the calcium salt is (2R,3S,5R)-5-(6-aminopurin-9-yl)-3-((cyclohexyloxy(oxy)phosphoryloxy)tetrahydrofuran-2-yl)methyl cyclohexyl phosphonate calcium salt. In some embodiments, the sodium salt is (2R,3S,5R)-5-(6-aminopurin-9-yl)-3-((cyclohexyloxy(oxy)phosphoryloxy)tetrahydrofuran-2-yl)methyl cyclohexyl phosphonate disodium salt. In some embodiments, the potassium salt is (2R,3S,5R)-5-(6-aminopurin-9-yl)-3-((cyclohexyloxy(oxy)phosphoryloxy)tetrahydrofuran-2-yl)methyl cyclohexyl phosphonate dipotassium salt. In some embodiments, the magnesium salt is (2R,3S,5R)-5-(6-aminopurin-9-yl)-3-((cyclohexyloxy(oxy)phosphoryloxy)tetrahydrofuran-2-yl)methyl cyclohexyl phosphonate magnesium salt. In some embodiments, the cobalt salt is (2R,3S,5R)-5-(6-aminopurin-9-yl)-3-((cyclohexyloxy(oxy)phosphoryloxy)tetrahydrofuran-2-yl)methyl cyclohexyl phosphonate cobalt salt.

Pharmaceutically acceptable salts of the present disclosure can be prepared by standard techniques known in the art to which the present disclosure pertains. For example, the free-base form of a compound of the present disclosure can be dissolved in a suitable solvent, such as an aqueous or aqueous-alcohol solution containing the appropriate acid and then isolated by evaporating the solution. In another example, a salt can be prepared by reacting the free base and acid in an organic solvent. If the particular compound is an acid, the desired pharmaceutically acceptable salt may be prepared by any suitable method, including, for example, treatment of the free acid with an appropriate inorganic or organic base.

In addition to the compound of Formula (I), the present disclosure also includes prodrugs (e.g., pharmaceutically acceptable prodrugs), active metabolic derivatives (active metabolites), and their pharmaceutically acceptable salts.

Prodrugs are compounds or pharmaceutically acceptable salts thereof which, when metabolized under physiological conditions or when converted by solvolysis, yield the desired active compound. Typically, the prodrug is inactive, or less active than the active compound, but may provide one or more advantageous handling, administration, and/or metabolic properties. Some prodrugs are activated enzymatically to yield the active compound, or a compound may undergo further chemical reaction to yield the active compound. Prodrugs may proceed from prodrug form to active form in a single step or may have one or more intermediate forms which such forms have activity or may be inactive.

As described in The Practice of Medicinal Chemistry, Ch. 31-32 (Ed. Wermuth, Academic Press, San Diego, Calif, 2001), prodrugs can be conceptually divided into two non-exclusive categories, including bioprecursor prodrugs and carrier prodrugs. Generally, bioprecursor prodrugs are compounds that are inactive or have low activity compared to the corresponding active drug compound that contain one or more protective groups and are converted to an active form by metabolism or solvolysis. Both the active drug form and any released metabolic products should have acceptably low toxicity. Typically, the formation of active drug compound involves a metabolic process or reaction that is one of the following types:

Oxidative reactions: Oxidative reactions are exemplified without limitation by reactions such as oxidation of alcohol, carbonyl, and acid functionalities, hydroxylation of aliphatic carbons, hydroxylation of alicyclic carbon atoms, oxidation of aromatic carbon atoms, oxidation of carbon-carbon double bonds, oxidation of nitrogen-containing functional groups, oxidation of silicon, phosphorus, arsenic, and sulfur, oxidative N-dealkylation, oxidative O- and S-dealkylation, oxidative deamination, as well as other oxidative reactions.

Reductive reactions: Reductive reactions are exemplified without limitation by reactions such as reduction of carbonyl functionalities, reduction of alcohol functionalities and carbon-carbon double bonds, reduction of nitrogen-containing functional groups, and other reduction reactions.

Reactions without change in the oxidation state: Reactions without change in the state of oxidation are exemplified without limitation by reactions such as hydrolysis of esters and ethers, hydrolytic cleavage of carbon-nitrogen single bonds, hydrolytic cleavage of non-aromatic heterocycles, hydration and dehydration at multiple bonds, new atomic linkages resulting from dehydration reactions, hydrolytic dehalogenation, removal of hydrogen halide molecule, and other such reactions.

Carrier prodrugs are drug compounds that contain a transport moiety, e.g., that improves uptake and/or localized delivery to a site(s) of action. Desirably for such a carrier prodrug, the linkage between the drug moiety and the transport moiety is a covalent bond, the prodrug is inactive or less active than the drug compound, and the prodrug and any release transport moiety are acceptably non-toxic. For prodrugs where the transport moiety is intended to enhance uptake, typically the release of the transport moiety should be rapid. In other cases, it is desirable to utilize a moiety that provides slow release, e.g., certain polymers or other moieties, such as cyclodextrins.

Such carrier prodrugs are often advantageous for orally administered drugs. In some instances, the transport moiety provides targeted delivery of the drug. For example, the drug may be conjugated to an antibody or antibody fragment. Carrier prodrugs can, for example, be used to improve one or more of the following properties: increased lipophilicity, increased duration of pharmacological effects, increased site-specificity, decreased toxicity and adverse reactions, and/or improvement in drug formulation (e.g., stability, water solubility, suppression of an undesirable organoleptic or physiochemical property). For example, lipophilicity can be increased by esterification of hydroxyl groups with lipophilic carboxylic acids, or of carboxylic acid groups with alcohols, e.g., aliphatic alcohols. Wermuth, supra.

Metabolites, e.g., active metabolites, overlap with prodrugs as described above, e.g., bioprecursor prodrugs. Thus, such metabolites are pharmacologically active compounds or compounds that further metabolize to pharmacologically active compounds that are derivatives resulting from metabolic processes in the body of a patient. Of these, active metabolites are such pharmacologically active derivative compounds. For prodrugs, the prodrug compound is generally inactive or of lower activity than the metabolic product. For active metabolites, the parent compound may be either an active compound or may be an inactive prodrug. For example, in some compounds, one or more alkoxy groups can be metabolized to hydroxyl groups while retaining pharmacologic activity and/or carboxyl groups can be esterified, e.g., glucuronidation. In some cases, there can be more than one metabolite, where an intermediate metabolite(s) is further metabolized to provide an active metabolite. For example, in some cases a derivative compound resulting from metabolic glucuronidation may be inactive or of low activity and can be further metabolized to provide an active metabolite.

Metabolites of a compound of the present disclosure may be identified using routine techniques known in the art, and their activities determined using tests such as those described in Bertolini et al., 1997, J. Med. Chem., 40:2011-2016; Shan et al., 1997, J Pharm Sci 86(7):756-757; Bagshawe, 1995, Drug Dev. Res., 34:220-230; Wermuth, supra.

It is understood by those skilled in the art that some compounds may exhibit tautomerism.

In such cases, the formulae provided herein expressly depict only one of the possible tautomeric forms. It is therefore to be understood that the compound of Formula (I) intends to represent any tautomeric form of the depicted compound and is not to be limited merely to the specific tautomeric form depicted by the drawing of the compound.

In the case of agents that are solids, it is understood by those skilled in the art that the compounds and salts may exist in different crystal or polymorphic forms, or may be formulated as co-crystals, or may be in an amorphous form, or may be any combination thereof (e.g. partially crystalline, partially amorphous, or mixtures of polymorphs) all of which are intended to be within the scope of the present disclosure and specified formulae. Whereas salts are formed by acid/base addition (i.e., a free base or free acid of the compound of interest forms an acid/base reaction with a corresponding addition base or addition acid, respectively, resulting in an ionic charge interaction), co-crystals are a new chemical species that is formed between neutral compounds, resulting in the compound and an additional molecular species in the same crystal structure.

In some instances, the compound of Formula (I) is complexed with an acid or a base, including without limitation base addition salts such as, for example, ammonium, diethylamine, ethanolamine, ethylenediamine, diethanolamine, t-butylamine, piperazine, meglumine; acid addition salts, such as, for example, acetate, acetylsalicylate, besylate, camsylate, citrate, formate, fumarate, glutarate, hydrochlorate, maleate, mesylate, nitrate, oxalate, phosphate, succinate, sulfate, tartrate, thiocyanate, and tosylate; and amino acids such as, for example, alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine or valine.

Additionally, the compound of Formula (I) is intended to cover hydrated or solvated forms, as well as unhydrated or unsolvated forms. Other examples of solvates include without limitation the compound of Formula (I) in combination with a suitable solvent, such as isopropanol, ethanol, methanol, DMSO, ethyl acetate, acetic acid or ethanolamine, and the like.

In some embodiments, a compound of the present disclosure is a protonated compound. As used herein, the term “protonated compound” refers to a compound of the present disclosure that is protonated by adding protons (or positively charged hydrogen ions) to proton acceptor sites of a compound of the present disclosure. In some embodiments, the proton acceptor sites include the phosphate groups of a compound of the present disclosure as well as any additional proton acceptor sites on either the ribose or the butyl groups of a compound of the present disclosure.

As the number of proton acceptor sites that are protonated on a compound of the present disclosure increases, the pH obtained when a compound of the present disclosure is dissolved in water having a pH of 7 decreases and thus the amount of protonation of a compound of the present disclosure can be determined by measuring the pH of solutions of water after addition of a compound of the present disclosure. pH indicates the hydrogen ion concentration of a solution.

Solutions with a high concentration of hydrogen ions have a low pH and are therefore acidic, whereas solutions with a low concentration of hydrogen ions have a high pH and are therefore basic. In some embodiments, the compounds of the present disclosure are protonated so that when dissolved in water (pH 7) such compounds form an aqueous solution having a pH of from less than about pH 7 to about pH 1. As used herein, the term “about,” when used with numerical values, is to be read as including the amount(s) specified and variations of 20%, 10%, 5%, 1%, 0.5%, and 0.1% of the amount specified. In some embodiments, a compound of the present disclosure is a protonated compound having a pH when dissolved in water of from less than about pH 6 to about pH 1. In some embodiments, a compound of the present disclosure is a protonated compound having a pH when dissolved in water of from about pH 5 to about pH 1. In some embodiments, a compound of the present disclosure is a protonated compound having a pH when dissolved in water of from about pH 4.5 to about pH 1. In some embodiments, a compound of the present disclosure is a protonated compound having a pH when dissolved in water of from about pH 4 to about pH 1. In some embodiments, a compound of the present disclosure is a protonated compound having a pH when dissolved in water of from about pH 3 to about pH 1. In some embodiments, a compound of the present disclosure is a protonated compound having a pH when dissolved in water of from about pH 2 to about pH 1. In some embodiments, a compound of the present disclosure is a protonated compound having a pH when dissolved in water of about pH 3 to about pH 5. In some embodiments, a compound of the present disclosure is a protonated compound having a pH when dissolved in water of about pH 3 to about pH 4.

In some embodiments, protonation can be accomplished by incubating a compound of the present disclosure in the presence of a strong acid. Although a compound of the present disclosure can be protonated by adding protons to the reactive sites on the compound, other modifications of a compound of the present disclosure are possible and are intended to be encompassed by the term protonated compound as used herein. In some embodiments, protonated forms of the compounds of the present disclosure can be generated by subjecting the purified, partially purified or crude compounds to a low pH (e.g., acidic) environment. In some embodiments, purified or crude compounds can be protonated with acid, including phosphoric acid, nitric acid, hydrochloric acid, and acetic acid.

Other procedures to prepare a protonated compound of the present disclosure known to the skilled artisan are equally contemplated to be within the scope of the present disclosure. In some embodiments, once the compounds of the present disclosure have been protonated, such compounds may be separated from any undesired components such as, for example, excess acid.

The skilled artisan would know of many ways to separate the compounds from undesired components, including but not limited to using an H+-cation exchanger (e.g., H+-SCX). In some embodiments, the compounds of the present disclosure may be subjected to chromatography following protonation. In some embodiments, a compound of the present disclosure is run over a poly(styrene-divinyl benzene) based resin (e.g., Hamilton's PRP-1 or 3 and Polymer Lab's PLRP) following protonation.

In some embodiments, the protonated compounds of the present disclosure can be used directly. In some embodiments, the protonated compounds of the present disclosure can be processed further to remove any excess acid or salt, e.g., via precipitation, reverse phase chromatography, diafiltration or gel filtration. In some embodiments, the protonated compounds of the present disclosure can be concentrated by lyophilization, solvent evaporation, and the like. In some embodiments, when suspended in water or saline, the compounds of the present disclosure generally exhibit a pH of from about pH 3 to about pH 5 depending upon the level of protonation/acidification, which is determined by how much acid is used in the acidification process. In some embodiments, compounds of the present disclosure can be protonated by passage over a cation exchange column charged with hydrogen ions.

In some embodiments, utilization of two butyl groups in a compound of the present disclosure prevents or limits substantial nuclease degradation, including without limitation exonuclease degradation, of a compound of the present disclosure. In some embodiments, the butyl groups are positioned to protect the ribose of a compound of the present disclosure. Percent acid degradation may be determined using analytical HPLC to assess the loss of functional molecules or by other suitable methods. Acid degradation is generally measured as a function of time. In some embodiments, the compounds of the present disclosure are also nuclease resistant, which allows such compounds to maintain activity (e.g., pH stability) in an in vivo setting. Percent degradation of the compounds of the present disclosure in a setting containing a nuclease may be determined by methods known to those skilled in the art, such as, for example, mass spectroscopy. Nuclease degradation is generally measured as a function of time. In some embodiments, a reference compound is employed in determining the extent or rate of acid or nuclease degradation. In some embodiments, the compounds of the present disclosure are 10%, 20%, 30%, 40%, 50%, 70%, 90%, 100%, 150%, 200%, 300%, 500% or 750% more stable than a reference compound.

A compound of the present disclosure in accordance with some embodiments is useful as an antiviral having activity against any virus. In some embodiments, the virus may be an enveloped virus. Examples of enveloped viruses include, but are not limited to: influenza, human cytomegalovirus (HCMV), HIV, respiratory syncytial virus (RSV), vaccinia virus, and human coronaviruses (such as NL63, 229E, OC43, and SARS-CoV-2). A non-limiting list of the viruses that a compound of the present disclosure is effective against include without limitation coronaviruses, human immunodeficiency virus (HIV), herpes simplex virus (HSV), cytomegalovirus (CMV), Hepatitis B virus (HBV), Hepatitis C virus (HCV), and influenza virus. A non-limiting list of the coronaviruses that a compound of the present disclosure is effective against include without limitation an alphacoronavirus, a betacoronavirus, a gammacoronavirus, a deltacoronavirus, an omicroncoronavirus, and a mixture of two or more thereof. A non-limiting list of the coronaviruses that a compound of the present disclosure is effective against include without limitation porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778 2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof. In some embodiments, a compound of the present disclosure is useful as an antiviral having activity against any variant of any virus disclosed herein.

In some embodiments, a compound of the present disclosure is useful in both therapeutic and non-therapeutic medical applications. In some embodiments including non-therapeutic medical applications, the antimicrobial effect of a compound of the present disclosure allows use of a compound of the present disclosure for sterilization (e.g., sterilization of a patient's skin or of a surface or an object, such as, for example, a surgical instrument), or sanitization (e.g., the cleansing of a surface, instrument, as to render the surface free of undesirable concentrations of disease-causing microorganisms). In some embodiments, the compounds of the present disclosure are effective in combating microbial contamination of laboratory cultures, consumables (e.g., food or beverage preparations), medical devices, hospital apparatus, or industrial processes. Therapeutic applications of a compound of the present disclosure are described herein.

The present disclosure also provides pharmaceutical compositions. As used herein, the term “pharmaceutical composition” refers to a pharmaceutical preparation that contains a compound of the present disclosure, or a pharmaceutically acceptable salt thereof, and is suitable for administration to a patient for therapeutic purposes. As used herein, the term “patient” refers to a living organism that is treated with a compound of the present disclosure, including without limitation any mammal such as, for example, humans, other primates (e.g., monkeys, chimpanzees, etc.), companion animals (e.g., dogs, cats, horses, etc.), farm animals (e.g., goats, sheep, pigs, cattle, etc.), laboratory animals (e.g., mice, rats, etc.), and wild and zoo animals (e.g., wolves, bears, deer, etc.).

In some embodiments, the pharmaceutical composition may include at least one pharmaceutically acceptable component to provide an improved formulation of a compound of the present disclosure, including without limitation one or more pharmaceutically acceptable carriers, excipients or diluents. The carrier, excipient or diluent may take a wide variety of forms depending on the form of preparation desired for administration.

As used herein, the term “carrier” includes without limitation calcium carbonate, calcium phosphate, various sugars, such as lactose, glucose, or sucrose, types of starch, cellulose derivatives, gelatin, lipids, liposomes, nanoparticles, physiologically acceptable liquids as solvents or for suspensions, including, for example, sterile solutions of water for injection (WFI), saline solution, dextrose solution, Hank's solution, Ringer's solution, vegetable oils, mineral oils, animal oils, polyethylene glycols, liquid paraffin, and the like.

As used herein, the term “excipient” generally includes without limitation fillers, binders, disintegrants, glidants, lubricants, complexing agents, solubilizers, stabilizer, preservatives, and surfactants, which may be chosen to facilitate administration of the compound by a particular route. Suitable excipients may also include, for example, colloidal silicon dioxide, silica gel, talc, magnesium silicate, calcium silicate, sodium aluminosilicate, magnesium trisilicate, powdered cellulose, macrocrystalline cellulose, carboxymethyl cellulose, cross-linked sodium carboxymethylcellulose, sodium benzoate, calcium carbonate, magnesium carbonate, stearic acid, aluminum stearate, calcium stearate, magnesium stearate, zinc stearate, sodium stearyl fumarate, syloid, stearowet C, magnesium oxide, starch, sodium starch glycolate, glyceryl monostearate, glyceryl dibehenate, glyceryl palmitostearate, hydrogenated vegetable oil, hydrogenated cotton seed oil, castor seed oil, mineral oil, polyethylene glycol (e.g., PEG 4000-8000), polyoxyethylene glycol, poloxamers, povidone, crospovidone, croscarmellose sodium, alginic acid, casein, methacrylic acid divinylbenzene copolymer, sodium docusate, cyclodextrins (e.g., 2-hydroxypropyl-delta-cyclodextrin), polysorbates (e.g., polysorbate 80), cetrimide, TPGS (d-alpha-tocopheryl polyethylene glycol 1000 succinate), magnesium lauryl sulfate, sodium lauryl sulfate, polyethylene glycol ethers, di-fatty acid ester of polyethylene glycols, or a polyoxyalkylene sorbitan fatty acid ester (e.g., polyoxyethylene sorbitan ester Tween®), polyoxyethylene sorbitan fatty acid esters, sorbitan fatty acid ester, e.g., a sorbitan fatty acid ester from a fatty acid such as oleic, stearic or palmitic acid, mannitol, xylitol, sorbitol, maltose, lactose, lactose monohydrate or lactose spray dried, sucrose, fructose, calcium phosphate, dibasic calcium phosphate, tribasic calcium phosphate, calcium sulfate, dextrates, dextran, dextrin, dextrose, cellulose acetate, maltodextrin, simethicone, polydextrosem, chitosan, gelatin, HPMC (hydroxypropyl methyl celluloses), HPC (hydroxypropyl cellulose), hydroxyethyl cellulose, and the like.

As the skilled artisan understands, any diluent known in the art may be utilized in accordance with the present disclosure. In some embodiments of the present disclosure, the diluent is water soluble. In some embodiments of the present disclosure, the diluent is water insoluble. As used herein, the term “diluent” includes without limitation water, saline, phosphate buffered saline (PBS), dextrose, glycerol, ethanol, buffered sodium or ammonium acetate solution, or the like, and combinations thereof.

In some embodiments, the pharmaceutical compositions of the present disclosure include at least one additional active ingredient. As used herein, the term “active ingredient” refers to a therapeutically active compound, as well as any prodrugs thereof and pharmaceutically acceptable salts, hydrates, and solvates of the compound and the prodrugs. Additional active ingredients may be combined with a compound of the present disclosure and may be either administered separately or in the same pharmaceutical composition. The amount of additional active ingredients to be given may be determined by one skilled in the art based upon therapy with a compound of the present disclosure.

In some embodiments, the pharmaceutical composition is a human pharmaceutical composition. As used herein, the term “human pharmaceutical composition” refers to a pharmaceutical composition intended for administration to a human.

The pharmaceutical compositions of the present disclosure are suitable for administration to a patient by any suitable means, including without limitation those means used to administer conventional antimicrobials. The pharmaceutical compositions of the present disclosure may be administered using any applicable route that would be considered by one of ordinary skill, including without limitation oral, intravenous (“IV”) injection or infusion, intravesical, subcutaneous (“SC”), intramuscular (“IM”), intraperitoneal, intradermal, intraocular, inhalation (and intrapulmonary), intranasal, transdermal, epicutaneously, subdermal, topical, mucosal, nasal, ophthalmic, impression into skin, intravaginal, intrauterine, intracervical, and rectal. Such dosage forms should allow a compound of the present disclosure to reach target cells. Other factors are well known in the art and include considerations such as toxicity and dosage forms that retard a compound or composition from exerting its effects. Techniques and formulations generally may be found in Remington: The Science and Practice of Pharmacy, 21st edition, Lippincott, Williams and Wilkins, Philadelphia, Pa., 2005.

In some embodiments, the pharmaceutical compositions of the present disclosure are adapted for topical administration. As used herein, the term “topical administration” refers to administration of a compound of the present disclosure to the skin surface of a patient so that a compound of the present disclosure passes through the skin layer. Transdermal administration and transmucosal administration are also encompassed within the term topical administration. As used herein, the term “transdermal” refers to passage of a compound of the present disclosure across at least one skin layer of a patient. As used herein, “transmucosal” refers to passage of a compound of the present disclosure across a mucous membrane of a patient. Unless otherwise stated or implied, the terms “topical administration,” “transdermal administration,” and “transmucal administration” are used interchangeably herein.

A variety of topical delivery systems for delivering bioactive compounds to microbes in a patient are well known in the art. Such systems include without limitation lotions, creams, gels, oils, ointments, solutions, suspensions, emulsions, and the like by choice of appropriate carriers in the art. In some embodiments, the pharmaceutical composition is administered in the form of a gel including a polyhydric alcohol.

Suitable carriers include without limitation vegetable or mineral oils, white petrolatum (e.g., white soft paraffin), branched chain fats or oils, animal fats and high molecular weight alcohol (e.g., greater than C12). In some embodiments, carriers are selected such that a compound of the present disclosure is soluble. In some embodiments, emulsifiers, stabilizers, humectants, and antioxidants may also be included as well as agents imparting color or fragrance, if desired. In some embodiments, an organic solvent or co-solvent such as ethanol or propanol may be employed in the pharmaceutical compositions of the present disclosure. In some embodiments, evaporation of the solvent leaves a residue on the treated surface to inhibit reinfection. In some embodiments, penetrants appropriate to the barrier to be permeated are used. Such penetrants are generally known in the art and include without limitation bile salts and fusidic acid derivatives. In some embodiments, detergents may be used to facilitate permeation. In some embodiments, creams for topical administration are formulated from a mixture of mineral oil, self-emulsifying beeswax, and water in which mixture a compound of the present disclosure, dissolved in a small amount of solvent (e.g., an oil), is admixed. The specific topical delivery system used depends on the location of the microbes.

In some embodiments, other materials may also be added to the topical pharmaceutical compositions of the present disclosure to have additional moisturizing effects and to improve the consistency of the pharmaceutical composition. Examples of such compounds include without limitation cetyl esters wax, stearyl alcohol, cetyl alcohol, glycerin, methyl paraben, propyl paraben, quaternium-15, humectants, volatile methylsiloxane fluids, and polydiorganosiloxane-polyoxyalkylene. See, e.g., U.S. Pat. Nos. 5,153,230 and 4,421,769. If it is desirable for the pharmaceutical composition to have additional cleaning effects in some embodiments, chemicals such as sodium lauryl sulfate or a metal salt of a carboxylic acid may be added.

In some embodiments, a wide variety of nonvolatile emollients are useful in the pharmaceutical compositions of the present disclosure. Non-limiting examples of such nonvolatile emollients are listed in McCutcheon's, Vol. 2 Functional Materials, North American Edition, (1992), pp. 137-168, and CTFA Cosmetic Ingredient Handbook, Second Edition (1992) which lists Skin-Conditioning Agents at pp. 572-575 and Skin Protectants at p. 580. In some embodiments, the nonvolatile emollients include silicones, hydrocarbons, esters, and mixtures thereof. In some embodiments, the esters include esters of monofunctional and difunctional fatty acids that have been esterified with alcohols and polyols (i.e., alcohols having two or more hydroxyl groups). In some embodiments, long chain esters of long chain fatty acids are utilized in the pharmaceutical compositions of the present disclosure (i.e., C10-40 fatty acids esterified with C10-40 fatty alcohols). Non-limiting examples of esters useful in the pharmaceutical compositions of the present disclosure include without limitation those selected from the group consisting of diisopropyl adipate, isopropyl myristate, isopropyl palmitate, myristyl propionate, ethylene glycol distearate, 2-ethylhexyl palmitate, isodecyl neopentanoate, C12-15 alcohol benzoate, di-2-ethylhexyl maleate, ceryl palmitate, myristyl myristate, stearyl stearate, cetyl stearate, behenyl behenrate, and mixtures thereof.

Examples of silicone emollients useful in the pharmaceutical compositions of the present disclosure include without limitation polyalkylsiloxanes, cyclic polyalkylsiloxanes, and polyalkylarylsiloxanes. Suitable commercially available polyalkylsiloxanes include the polydimethylsiloxanes, which are also known as dimethicones, non-limiting examples of which include the Vicasil™ series (sold by General Electric Company) and the Dow Corning™ 200 series (sold by Dow Corning Corporation). Commercially available polyalkylsiloxanes include cyclomethicones, non-limiting examples of which include Dow Corning™ 244 fluid, Dow Corning™ 344 fluid, Dow Corning™ 245 fluid, and Dow Corning™ 345, among others. A suitable commercially available trimethylsiloxysilicate is sold as a mixture with dimethicone as Dow Corning™ 593 fluid. Also useful in the pharmaceutical compositions of the present disclosure are dimethiconols, which are hydroxyl terminated dimethyl silicones. Suitable commercially available dimethiconols are typically sold as mixtures with dimethicone or cyclomethicone (e.g., Dow Corning™ 1401, 1402, and 1403 fluids). Suitable commercially available polyalkylarylsiloxanes include SF1075 methylphenyl fluid (sold by General Electric Company) and 556 Cosmetic Grade phenyl trimethicone fluid (sold by Dow Corning Corporation).

Hydrocarbons suitable for use in the pharmaceutical compositions of the present disclosure include without limitation straight and branched chain hydrocarbons having from about 10 to about 30 carbon atoms. In some embodiments, the straight and branched chain hydrocarbons have from about 12 to about 24 carbon atoms. In some embodiments, the straight and branched chain hydrocarbons have from about 16 to about 22 carbon atoms. Non-limiting examples of such hydrocarbon materials include dodecane, squalane, cholesterol, 5 hydrogenated polyisobutylene, docosane (i.e., a C22 hydrocarbon), hexadecane, and isohexadecane (a commercially available hydrocarbon sold as Permethyl™ 101A by Presperse, South Plainsfield, N.J.), among others.

In some embodiments, the topical pharmaceutical compositions of the present disclosure include propylene glycol. In some embodiments, propylene glycol acts as a surfactant and assists in penetration, contact, and absorption of a compound of the present disclosure. In some embodiments, propylene glycol serves as a preservative. In some embodiments, the pharmaceutical compositions of the present disclosure include a non-ionic surfactant, such as, for example, polysorbate. Such a surfactant provides better surface contact of the pharmaceutical compositions of the present disclosure with mucosa (such as vaginal mucosa) by further reducing surface tension.

The topical pharmaceutical compositions of the present disclosure optionally may also be formulated with a lipophilic phase, such as, for example, emulsions and liposome dispersions. In some embodiments, liposomal formulations may extend circulation time of a compound of the present disclosure, increase permeability of a compound of the present disclosure, and improve overall efficacy of a compound of the present disclosure as an antimicrobial. In some embodiments, a compound of the present disclosure may be combined with a lipid, cationic lipid or anionic lipid. In some embodiments, the resulting emulsion or liposomal suspension in conjunction with the pH stabilizing qualities of a compound of the present disclosure can effectively increase the in vivo half-life of the activity of a pharmaceutical composition of the present disclosure. Examples of suitable anionic lipids for use with the pharmaceutical compositions of the present disclosure include, but are not limited to, cardiolipin, dimyristoyl, dipalmitoyl, dioleoyl phosphatidyl choline, phosphatidyl glycerol, palmitoyloleoyl phosphatidyl choline, phosphatidyl glycerol, phosphatidic acid, lysophosphatidic acid, phosphatidyl serine, phosphatidyl inositol, and anionic forms of cholesterol.

In some embodiments, a compound of the present disclosure is incorporated into liposomes. In some embodiments, neutral lipids, cholesterol, and/or polyethylene glycol (PEG) are utilized in such liposomes. In some embodiments, the liposomal composition is composed of partially hydrogenated soy phosphatidylcholine (PHSC), cholesterol, methoxy-terminated PEG (mPEG), and/or distearoyl phosphatidyl ethanolamine (DSPE). The liposomes can be prepared according to any suitable method known in the art.

In some embodiments, topical administration is through nasal sprays or suppositories (rectal or vaginal). Suppositories are prepared by mixing a compound of the present disclosure with a lipid vehicle such as Theobroma oil, cacao butter, glycerin, gelatin, polyoxyethylene glycols, and the like. In some embodiments, topical administration comprises a transdermal patch or dressing such as a bandage impregnated with a compound of the present disclosure and optionally one or more carriers, excipients or diluents known in the art. In some embodiments, such dressings include without limitation semipermeable films, foams, hydrocolloids, and calcium alginate swabs. In some embodiments, the dosage administration will be continuous rather than intermittent throughout the dosage regimen.

In some embodiments, the pharmaceutical compositions of the present disclosure are adapted for oral administration. As used herein, the term “oral administration” refers to administration of a compound of the present disclosure to the mouth of a patient for ingestion into the gastrointestinal tract. In some embodiments, the pharmaceutical compositions of the present disclosure can be formulated into conventional oral dosage forms including without limitation capsules, tablets, powders, and liquid preparations such as suspensions, solutions, elixirs, syrups, concentrated drops, and the like. In some embodiments, a compound of the present disclosure may be combined with solid excipients, optionally grinding a resulting mixture, and optionally processing the mixture of granules, after adding suitable auxiliaries, if desired, to obtain, for example, tablets, coated tablets, hard capsules, soft capsules, solutions (e.g., aqueous, alcoholic or oily solutions), and the like. In some embodiments, excipients suitable for use in the oral pharmaceutical compositions of the present disclosure include without limitation fillers such as sugars, including lactose, glucose, sucrose, mannitol, or sorbitol; cellulose preparations, for example, corn starch, wheat starch, rice starch, potato starch, gelatin, gum tragacanth, methyl cellulose, hydroxypropylmethyl-cellulose, sodium carboxymethylcellulose (CMC), and/or polyvinylpyrrolidone (PVP or povidone); and oily excipients, including vegetable and animal oils, such as sunflower oil, olive oil, or cod liver oil. In some embodiments, the oral pharmaceutical compositions of the present disclosure may also contain disintegrating agents, such as, for example, cross-linked polyvinylpyrrolidone, agar, or alginic acid, or a salt thereof, such as sodium alginate; a lubricant, such as talc or magnesium stearate; a plasticizer, such as glycerol or sorbitol; a sweetening agent such as sucrose, fructose, lactose, or aspartame; a natural or artificial flavoring agent, such as, for example, peppermint, oil of wintergreen, or cherry flavoring; or dye-stuffs or pigments, which may be used for identification or characterization of different doses or combinations. In some embodiments, the oral pharmaceutical compositions of the present disclosure may also contain dragée cores with suitable coatings. In some embodiments, concentrated sugar solutions may be used, which may optionally contain, for example, gum arabic, talc, poly-vinylpyrrolidone, carbopol gel, polyethylene glycol, titanium dioxide, lacquer solutions, and suitable organic solvents or solvent mixtures.

In some embodiments, the pharmaceutical compositions of the present disclosure that can be used orally include without limitation push-fit capsules made of gelatin (“gelcaps”), as well as soft, sealed capsules made of gelatin, and a plasticizer, such as glycerol or sorbitol. In some embodiments, the push-fit capsules can contain a compound of the present disclosure in admixture with fillers such as lactose, binders such as starches, and/or lubricants such as talc or magnesium stearate and, optionally, stabilizers. In some embodiments, including soft capsules, the active compound may be dissolved or suspended in suitable liquids, such as fatty oils, liquid paraffin, liquid polyethylene glycols, and the like.

In some embodiments, the pharmaceutical compositions of the present disclosure are adapted for inhalation administration. As used herein, the term “inhalation administration” refers to delivery of a compound of the present disclosure by passage through a patient's nose or mouth during inhalation and passage of the compound through the walls of the lungs of the patient. In some embodiments, the pharmaceutical compositions of the present disclosure suitable for inhalation administration may be formulated as dry powder or a suitable solution, suspension or aerosol. In some embodiments, powders and solutions may be formulated with suitable additives known in the art. In some embodiments, powders may include a suitable powder base such as lactose or starch. In some embodiments, solutions may comprise propylene glycol, sterile water, ethanol, sodium chloride, and other additives, such as, for example, acid, alkali, and buffer salts. In some embodiments, such solutions or suspensions may be administered by inhaling via a spray, pump, atomizer, nebulizer, and the like. In some embodiments, the pharmaceutical compositions of the present disclosure suitable for inhalation administration may also be used in combination with other inhaled therapies, including without limitation corticosteroids such as, for example, fluticasone proprionate, beclomethasone dipropionate, triamcinolone acetonide, budesonide, and mometasone furoate; beta agonists such as, for example, albuterol, salmeterol, and formoterol; anticholinergic agents such as, for example, ipratroprium bromide or tiotropium; vasodilators such as, for example, treprostinal and iloprost; enzymes such as, for example, DNAase; therapeutic proteins; immunoglobulin antibodies; an oligonucleotide, such as, for example, single or double stranded DNA or RNA, siRNA; antibiotics such as, for example, tobramycin; muscarinic receptor antagonists; leukotriene antagonists; cytokine antagonists; protease inhibitors; cromolyn sodium; nedocril sodium; and sodium cromoglycate.

In some embodiments, the pharmaceutical compositions of the present disclosure are adapted for intravesical administration. As used herein, the term “intravesical administration” refers to delivery of a compound of the present disclosure directly into the bladder of a patient. In some embodiments, the pharmaceutical composition is administered via a catheter. In some embodiments, the catheter is a urethral catheter.

In some embodiments, the pharmaceutical compositions of the present disclosure are adapted for parenteral administration. As used herein, the term “parenteral administration” refers to a compound of the present disclosure being injected or infused into a patient and includes without limitation intravenous, intramuscular, intraarterial, intrathecal, intraventricular, intracapsular, intraorbital, intracardiac, intradermal, intraperitoneal, transtracheal, subcutaneous, subcuticular, intraarticular, sub capsular, subarachnoid, intraspinal, intracerebro spinal, and intrasternal injection and infusion. In some embodiments, the pharmaceutical compositions of the present disclosure suitable for parenteral administration may be formulated in sterile liquid solutions, including without limitation physiologically compatible buffers or solutions, such as, for example, saline solution, Hank's solution or Ringer's solution. In some embodiments, the pharmaceutical compositions of the present disclosure suitable for parenteral administration may be prepared as dispersions in non-aqueous solutions, such as, for example, glycerol, propylene glycol, ethanol, liquid polyethylene glycols, triacetin, vegetable oils, and the like. In some embodiments, solutions may also contain a preservative, such as, for example, methylparaben, propylparaben, chlorobutanol, phenol, sorbic acid, thimerosal, and the like. In addition, pharmaceutical compositions of the present disclosure suitable for parenteral administration may be formulated in solid form, including, for example, lyophilized forms, and redissolved or suspended prior to use. In some embodiments, the pharmaceutical composition is administered via a needle.

The present disclosure also provides methods of treatment. As used herein, the terms “treating,” “treatment,” “therapy,” and like terms refer to administration of a compound or pharmaceutical composition of the present disclosure in an amount effective to prevent, alleviate or ameliorate one or more symptoms of a disease or condition (i.e., indication) and/or to prolong the survival of the patient being treated. In some embodiments, “treating,” “treatment,” “therapy,” and like terms also include without limitation reducing or eliminating infection in a patient.

In carrying out the methods of the present disclosure, an effective amount of a compound of the present disclosure is administered to a patient in need thereof. As used herein, the term “effective amount,” in the context of administration, refers to the amount of a compound or pharmaceutical composition of the present disclosure that, when administered to a patient, is sufficient to prevent, alleviate or ameliorate one or more symptoms of a disease or condition (i.e., indication) and/or to prolong the survival of the patient being treated. Such an amount should result in no or few adverse events in the treated patient. Similarly, such an amount should result in no or few toxic effects in the treated patient. As those familiar with the art will understand, the amount of a compound or pharmaceutical composition of the present disclosure will vary depending upon a number of factors, including without limitation the activity of a compound of the present disclosure (in vitro, e.g. a compound of the present disclosure vs. target, or in vivo activity in animal efficacy models), pharmacokinetic results in animal models (e.g., biological half-life or bioavailability), the type of patient being treated, the patient's age, size, weight, and general physical condition, the disorder associated with the patient, and the dosing regimen being employed in the treatment.

In some embodiments of the present disclosure, an effective amount of a compound of the present disclosure to be delivered to a patient in need thereof can be quantified by determining micrograms of a compound of the present disclosure per kilogram of patient body weight. In some embodiments, the amount of a compound of the present disclosure administered to a patient is from about 0.1 to about 1000 milligram (mg) of a compound of the present disclosure per kilogram (kg) of patient body weight. In some embodiments, the amount of a compound of the present disclosure administered to a patient is from about 0.1 to about 500 mg of a compound of the present disclosure per kg of patient body weight. In some embodiments, the amount of a compound of the present disclosure administered to a patient is from about 0.1 to about 300 mg of a compound of the present disclosure per kg of patient body weight. In some embodiments, the amount of a compound of the present disclosure administered to a patient is from about 0.1 to about 200 mg of a compound of the present disclosure per kg of patient body weight. In some embodiments, the amount of a compound of the present disclosure administered to a patient is from about 0.1 to about 100 mg of a compound of the present disclosure per kg of patient body weight. As those of ordinary skill in the art understand, multiple doses may be used.

In some embodiments of the present disclosure, a compound of the present disclosure is administered as a multiple dose regimen. As used herein, the term “multiple dose regimen” refers to a treatment time period of more than one day. In some embodiments of the present disclosure, the multiple dose regimen is a time period of up to about 2 days. In some embodiments of the present disclosure, the multiple dose regimen is a time period of up to about 3 days. In some embodiments of the present disclosure, the multiple dose regimen is a time period of up to about 4 days. In some embodiments of the present disclosure, the multiple dose regimen is a time period of up to about 5 days. In some embodiments of the present disclosure, the multiple dose regimen is a time period of up to about 6 days. In some embodiments of the present disclosure, the multiple dose regimen is a time period of up to about 7 days. In some embodiments of the present disclosure, the multiple dose regimen is a time period of up to about 14 days. In some embodiments of the present disclosure, the multiple dose regimen is a time period of up to about one month. In some embodiments of the present disclosure, the multiple dose regimen is a time period of up to about two months. In some embodiments of the present disclosure, the multiple dose regimen is a time period of up to about three months. In some embodiments of the present disclosure, the multiple dose regimen is a time period of up to about four months. In some embodiments of the present disclosure, the multiple dose regimen is a time period of up to about five months. In some embodiments of the present disclosure, the multiple dose regimen is a time period of up to about six months. Other time periods may be used herein.

In some embodiments of the present disclosure, a compound of the present disclosure is administered as part of a chronic treatment regimen. As used herein, the term “chronic treatment regimen” refers to treatment with a compound of the present disclosure over an extended period of time during a patient's lifetime. In some embodiments, chronic treatment is lifelong treatment.

In some embodiments of the present disclosure, a compound of the present disclosure is administered as a single dose. In some embodiments of the present disclosure, a compound of the present disclosure is administered as a single unit dose. As used herein, the term “unit dose” is a predetermined amount of a compound of the present disclosure. The amount of a compound of the present disclosure is generally equal to the dosage of a compound of the present disclosure that would be administered to a patient or a convenient fraction of such a dosage such as, for example, one-half or one-third of such a dosage. According to the methods of the present disclosure, the terms “single dose” and “single unit dose” include embodiments wherein a compound of the present disclosure can be administered as a single application and administered as multiple applications.

In some embodiment, a compound of the present disclosure may also be used in combination with one or more additional active ingredients for treating the same disease or condition. In some embodiments, such combination use includes administration of a compound of the present disclosure and one or more additional active ingredient at different times, or co-administration of a compound of the present disclosure and one or more additional active ingredients. In some embodiments, dosage may be modified for a compound of the present disclosure, or one or more additional active ingredients used in combination, e.g., reduction in the amount dosed relative to a compound of the present disclosure or one or more additional active ingredients used alone, by methods well known to those of ordinary skill in the art. In some embodiments, co-administration includes simultaneous administration of a compound of the present disclosure and an additional active ingredient in the same dosage form, simultaneous administration of a compound of the present disclosure and an additional active ingredient in separate dosage forms, and separate administration of a compound of the present disclosure and an additional active ingredient.

It is understood that use in combination includes use with one or more additional active ingredients or other medical procedure in which the one or more additional active ingredients or other medical procedure may be administered at different times (e.g., within a short time, such as within hours (e.g., 1, 2, 3, 4-24 hours, etc.), or within a longer time (e.g., 1-2 days, 2-4 days, 4-7 days, 1-4 weeks, etc.)) than a compound or pharmaceutical composition of the present disclosure, or at the same time as a compound or pharmaceutical composition of the present disclosure. Use in combination also includes use with one or more additional active ingredients or other medical procedure that is administered once or infrequently, such as surgery, along with a compound or pharmaceutical composition of the present disclosure administered within a short time or longer time before or after the administration of the one or more additional active ingredients or completion of the other medical procedure.

In some embodiments, the present disclosure provides for delivery of a compound or pharmaceutical composition of the present disclosure and one or more additional active ingredients delivered by a different route of administration or by the same route of administration. In some embodiments, the use in combination for any route of administration includes delivery of a compound or pharmaceutical composition of the present disclosure and one or more additional active ingredients delivered by the same route of administration together in any pharmaceutical composition, including pharmaceutical compositions in which the two compounds are chemically linked in such a way that such compounds maintain their therapeutic activity when administered. In some embodiments, the one or more additional active ingredients may be co-administered with a compound or pharmaceutical composition of the present disclosure. In some embodiments, use in combination by co-administration includes administration of co-formulations or formulations of chemically joined compounds, or administration of two or more compounds in separate formulations within a short time of each other (e.g., within an hour, 2 hours, 3 hours, up to 24 hours, etc.), administered by the same or different routes. In some embodiments, co-administration of separate formulations includes co-administration by delivery via one device, for example, the same inhalant device, the same syringe, etc., or administration from separate devices within a short time of each other. In some embodiments, co-formulations of a compound or pharmaceutical composition of the present disclosure and one or more additional active ingredients delivered by the same route includes preparation of the materials together such that they can be administered by one device, including the separate compounds combined in one formulation, or compounds that are modified such that the compounds are chemically joined, yet still maintain their biological activity. In some embodiments, such chemically joined compounds may have a linkage that is substantially maintained in vivo, or the linkage may break down in vivo, separating the two active components.

The present disclosure also provides a method of treating an infection in a patient in need thereof. In some embodiments, the method comprises administering an effective amount of a compound of the present disclosure, or a pharmaceutically acceptable salt thereof, to the patient. As used herein, the term “infection” refers to any microbe infection of a patient's body. Infection includes the invasion of a patient's body by a microbe and subsequent multiplication in the patient's body.

The present disclosure also provides a kit. In some embodiments, the kit comprises a compound according to the present disclosure, or a pharmaceutically acceptable salt thereof, or a pharmaceutical composition according to the present disclosure. As used herein, the term “kit” refers to any manufacture, such as, for example, a package, container, and the like, containing a compound according to the present disclosure, or a pharmaceutically acceptable salt thereof, or a pharmaceutical composition according to the present disclosure. In some embodiments, a compound according to the present disclosure, or a pharmaceutically acceptable salt thereof, or a pharmaceutical composition according to the present disclosure is packaged in a vial, bottle, tube, flask or patch, which may be further packaged within a box, envelope, bag, or the like. In some embodiments, a compound according to the present disclosure, or a pharmaceutically acceptable salt thereof, or a pharmaceutical composition according to the present disclosure is approved by the U.S. Food and Drug Administration or similar regulatory agency in the U.S. or a jurisdiction or territory outside the U.S. for administration to a patient. In some embodiments, the kit includes written instructions for use and/or other indication that a compound according to the present disclosure, or a pharmaceutically acceptable salt thereof, or a pharmaceutical composition according to the present disclosure is suitable or approved for administration to a patient. In some embodiments, a compound or composition of the present disclosure is packaged in unit dose or single unit dose form, such as, for example, single unit dose pills, capsules or the like. In some embodiments, the kit includes a dispenser.

The present disclosure also provides the use of a compound of the present disclosure, or a pharmaceutically acceptable salt thereof, for the manufacture of a medicament. As used herein, the term “medicament” refers to a pharmaceutical composition according to the present disclosure.

In some embodiments, the pharmaceutical composition is contained in any manufacture, such as, for example, a package, container, and the like.

In addition to the aspects and embodiments described and provided elsewhere in the present disclosure, the following non-limiting list of embodiments are also contemplated.
1. A method for treating a coronavirus infection in a patient in need thereof, the method comprising:
administering an effective amount of a composition comprising a compound having the formula):

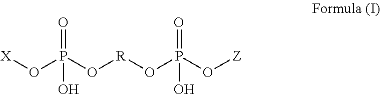

wherein:
   Z and X are independently selected from alkyl, alkyloxyalkyl, alkylaryl, aryl, alkenyl, arylalkyl, arylalkenyl, or alkylarylalkenyl, and each substituent, Z or X, is optionally substituted with other functional groups; and
   R is selected from alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and each substituent, R, is optionally substituted with other functional groups; or
a pharmaceutically acceptable salt thereof.
2. The method of clause 1, wherein the coronavirus is selected from the group consisting of an alphacoronavirus, a betacoronavirus, a gammacoronavirus, a deltacoronavirus, and an omicroncoronavirus, or combinations thereof.
3. The method of clause 1 or clause 2, wherein the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhlii coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.
4. The method of any one of clauses 1 to 3, wherein the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, and bat Hp-betacoronavirus Zhejiang2013, or combinations thereof.
5. The method of any one of clauses 1 to 4, wherein the coronavirus is human SARS-CoV-2.
6. The method of any one of clauses 1 to 5, wherein the coronavirus is human SARS-CoV.
7. The method of any one of clauses 1 to 6, wherein the compound comprises a compound having the formula:

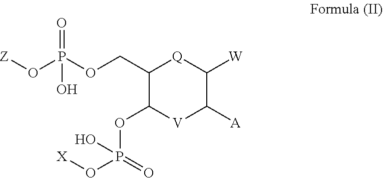

wherein:
   Z and X are independently selected from alkyl, alkylaryl, aryl, alkenyl, arylalkyl, arylalkenyl, or alkylarylalkenyl, and each substituent, Z or X, is optionally substituted with other functional groups;
   Q is O or S;
   V is a bond, O, or S;
   A is H, alkyl, or alkyloxy, and A is optionally substituted with other functional groups; and
   W is a monocyclic or bicyclic heterocycle; or
a pharmaceutically acceptable salt thereof.
8. The method of clause 7, wherein the bond to W is to a heteroatom of the heterocycle.
9. The method of clause 7, wherein the bond to W is to a carbon atom of the heterocycle.
10. The method of any one of clauses 1 to 9, wherein the compound comprises a compound having the formula:

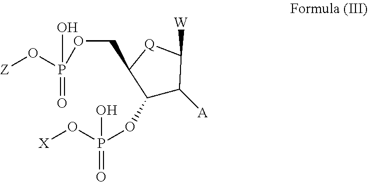

wherein:
   Z and X are independently selected from alkyl, alkylaryl, aryl, alkenyl, arylalkyl, arylalkenyl, or alkylarylalkenyl, and each substituent, Z or X, is optionally substituted with other functional groups;
   Q is O or S;
   V is a bond, O, or S;
   A is H, alkyl, or alkyloxy, and A is optionally substituted with other functional groups; and
   W is a monocyclic or bicyclic heterocycle; or
a pharmaceutically acceptable salt thereof.
11. The method of clause 10, wherein the bond to W is to a heteroatom of the heterocycle.
12. The method of clause 10, wherein the bond to W is to a carbon atom of the heterocycle.
13. The method of any one of clauses 1 to 12, wherein Z and X are independently alkyl and are substituted with at least one hydroxyl group.
14. The method of any one of clauses 1 to 13, wherein Z and X are both 4-hydroxybutyl.
15. The method of any one of clauses 1 to 12, wherein Z and X are both n-butyl.
16. The method of any one of clauses 1 to 6, wherein the compound is selected from the group consisting of a compound having the formula:

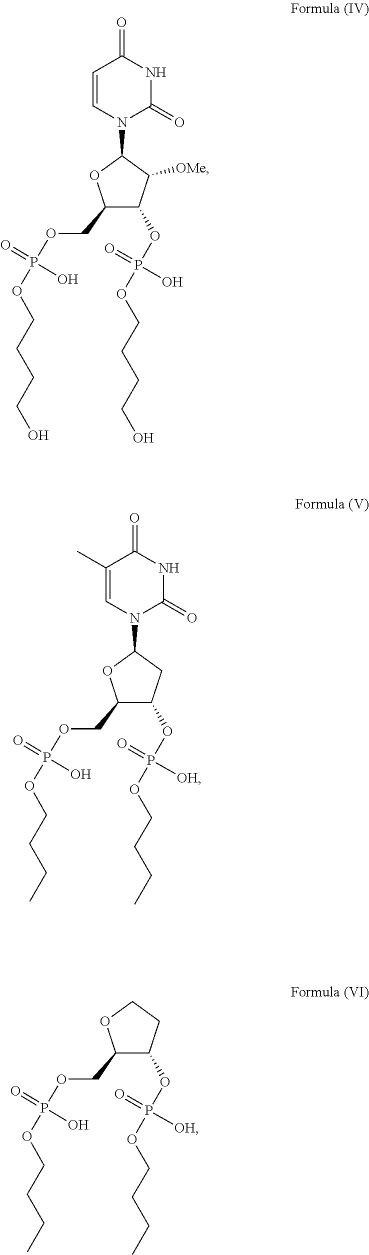 [SMILES: CO[C@@H]1[C@H](OP(=O)(O)OCCCCO)[C@@H](COP(=O)(O)OCCCCO)O[C@H]1n1ccc(=O)[nH]c1=O ; CCCCOP(=O)(O)OC[C@H]1O[C@@H](n2cc(C)c(=O)[nH]c2=O)C[C@@H]1OP(=O)(O)OCCCC ; CCCCOP(=O)(O)OC[C@H]1OCC[C@@H]1OP(=O)(O)OCCCC]

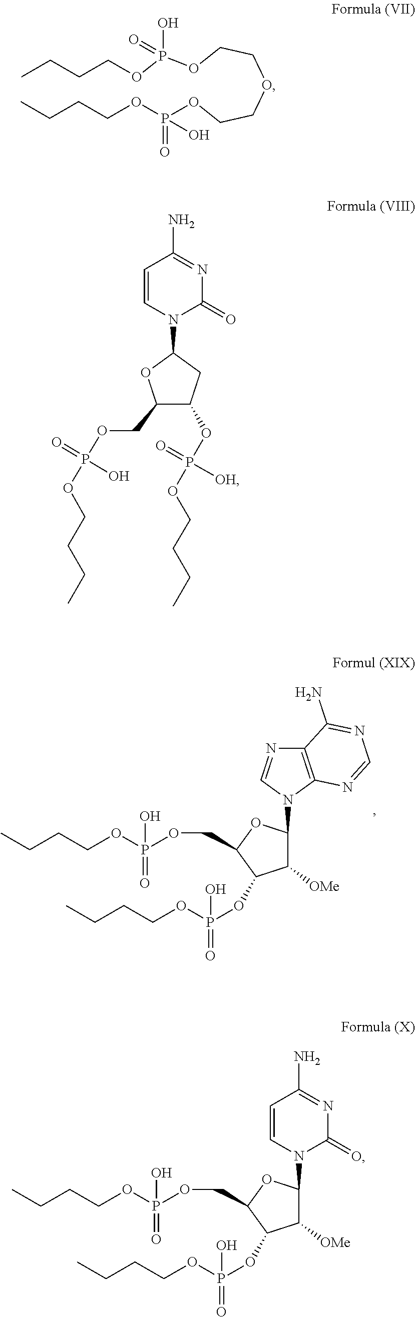 [SMILES: CCCCOP(=O)(O)OCCOCCOP(=O)(O)OCCCC ; CCCCOP(=O)(O)OC[C@H]1O[C@@H](n2ccc(N)nc2=O)C[C@@H]1OP(=O)(O)OCCCC ; CCCCOP(=O)(O)OC[C@H]1O[C@@H](n2cnc3c(N)ncnc32)[C@H](OC)[C@@H]1OP(=O)(O)OCCCC ; CCCCOP(=O)(O)OC[C@H]1O[C@@H](n2ccc(N)nc2=O)[C@H](OC)[C@@H]1OP(=O)(O)OCCCC]

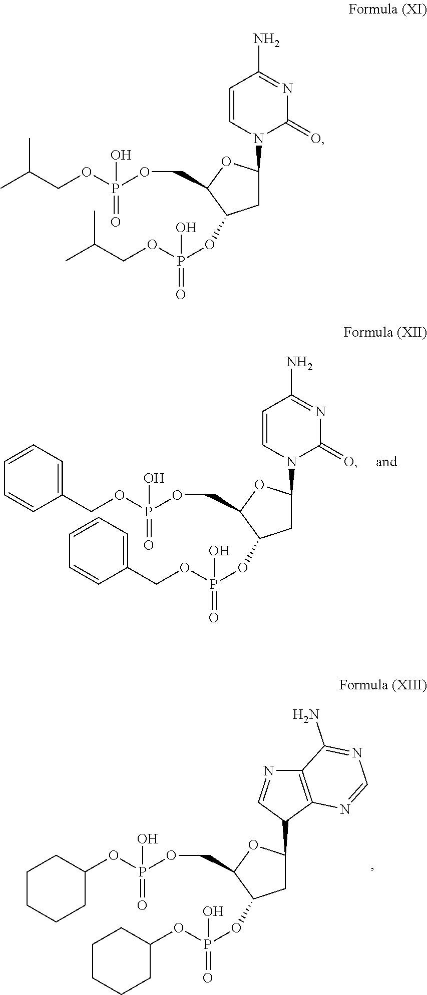 [SMILES: CC(C)COP(=O)(O)OC[C@H]1O[C@@H](n2ccc(N)nc2=O)C[C@@H]1OP(=O)(O)OCC(C)C ; Nc1ccn([C@H]2C[C@H](OP(=O)(O)OCc3ccccc3)[C@@H](COP(=O)(O)OCc3ccccc3)O2)c(=O)n1 ; "and" ; Nc1ncnc2c1N=CC2[C@H]1C[C@H](OP(=O)(O)OC2CCCCC2)[C@@H](COP(=O)(O)OC2CCCCC2)O1]

or combinations thereof; or
a pharmaceutically acceptable salt thereof.
17. The method of any one of clauses 1 to 16, wherein the compound comprises a compound having the formula:

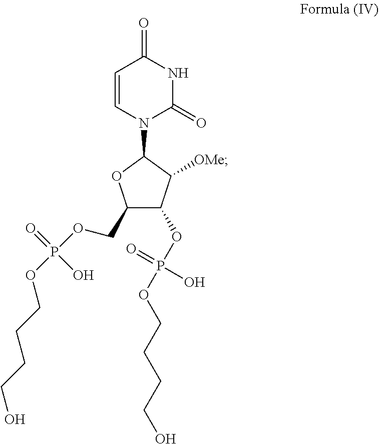 [SMILES: CO[C@@H]1[C@H](OP(=O)(O)OCCCCO)[C@@H](COP(=O)(O)OCCCCO)O[C@H]1n1ccc(=O)[nH]c1=O]

or a pharmaceutically acceptable salt thereof.
18. The method of any one of clauses 1 to 16, wherein the compound comprises a compound having the formula:

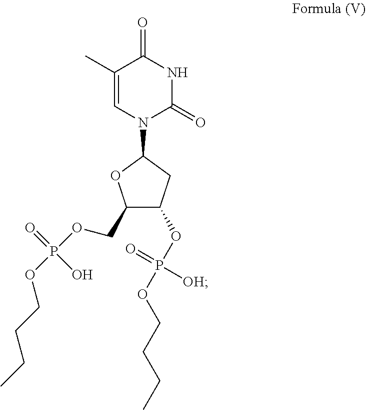 [SMILES: CCCCOP(=O)(O)OC[C@H]1O[C@@H](n2cc(C)c(=O)[nH]c2=O)C[C@@H]1OP(=O)(O)OCCCC]

or a pharmaceutically acceptable salt thereof.
19. The method of any one of clauses 1 to 16, wherein the compound comprises a compound having the formula:

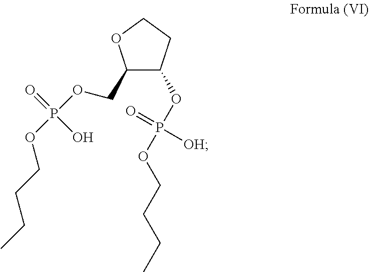 [SMILES: CCCCOP(=O)(O)OC[C@H]1OCC[C@@H]1OP(=O)(O)OCCCC]

or a pharmaceutically acceptable salt thereof.
20. The method of any one of clauses 1 to 6, wherein the compound comprises a compound having the formula:

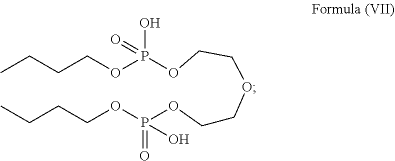 [SMILES: CCCCOP(=O)(O)OCCOCCOP(=O)(O)OCCCC]

or a pharmaceutically acceptable salt thereof.
21. The method of any one of clauses 1 to 16, wherein the compound comprises a compound having the formula:

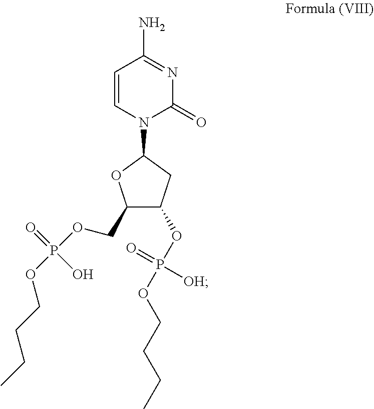 [SMILES: CCCCOP(=O)(O)OC[C@H]1O[C@@H](n2ccc(N)nc2=O)C[C@@H]1OP(=O)(O)OCCCC]

or a pharmaceutically acceptable salt thereof.
22. The method of any one of clauses 1 to 16, wherein the compound comprises a compound having the formula:

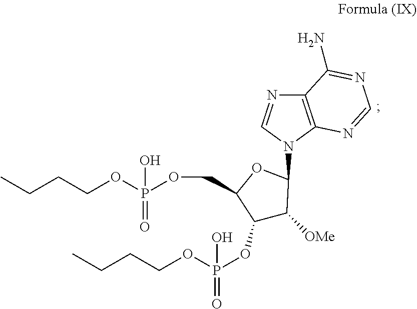 [SMILES: CCCCOP(=O)(O)OC[C@H]1O[C@@H](n2cnc3c(N)ncnc32)[C@H](OC)[C@@H]1OP(=O)(O)OCCCC]

or a pharmaceutically acceptable salt thereof.
23. The method of any one of clauses 1 to 16, wherein the compound comprises a compound having the formula:

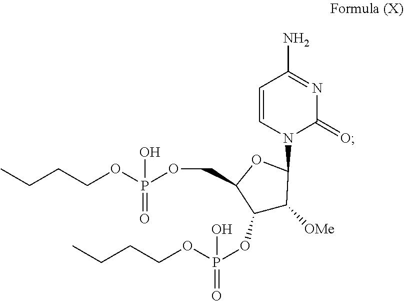 [SMILES: CCCCOP(=O)(O)OC[C@H]1O[C@@H](n2ccc(N)nc2=O)[C@H](OC)[C@@H]1OP(=O)(O)OCCCC]

or a pharmaceutically acceptable salt thereof.
24. The method of any one of clauses 1 to 16, wherein the compound comprises a compound having the formula:

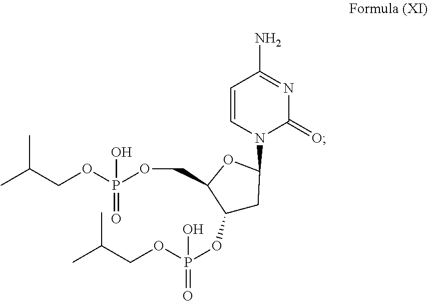 [SMILES: CC(C)COP(=O)(O)OC[C@H]1O[C@@H](n2ccc(N)nc2=O)C[C@@H]1OP(=O)(O)OCC(C)C]

or a pharmaceutically acceptable salt thereof.
25. The method of any one of clauses 1 to 16, wherein the compound comprises a compound having the formula:

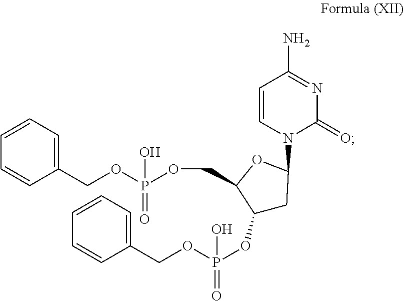 [SMILES: Nc1ccn([C@H]2C[C@H](OP(=O)(O)OCc3ccccc3)[C@@H](COP(=O)(O)OCc3ccccc3)O2)c(=O)n1]

or a pharmaceutically acceptable salt thereof.
26. The method of any one of clauses 1 to 16, wherein the compound comprises a compound having the formula:

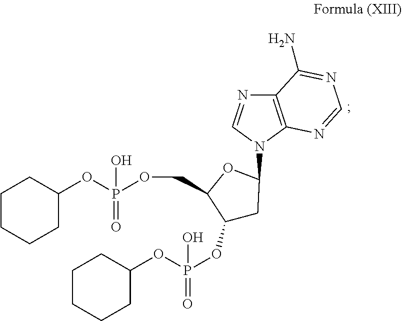 [SMILES: Nc1ncnc2c1ncn2[C@H]1C[C@H](OP(=O)(O)OC2CCCCC2)[C@@H](COP(=O)(O)OC2CCCCC2)O1]

or a pharmaceutically acceptable salt thereof.
27. The method of any one of clauses 1 to 26, wherein the pharmaceutically acceptable salt is selected from the group consisting of an ammonium salt, a calcium salt, a sodium salt, a potassium salt, a magnesium salt, and a cobalt salt, or combinations thereof.
28. The method of any one of clauses 1 to 27, wherein the compound, or the pharmaceutically acceptable salt thereof, is a protonated compound having a pH when dissolved in water of about pH 3 to about pH 5.
29. The method of any one of clauses 1 to 28, wherein the compound, or the pharmaceutically acceptable salt thereof, is a protonated compound having a pH when dissolved in water of about pH 3 to about pH 4.
30. The method of any one of clauses 1 to 29, wherein the composition further comprises one or more pharmaceutically acceptable carriers, excipients, or diluents.
31. The method of any one of clauses 1 to 30, wherein the composition further comprises at least one additional active ingredient.
32. The method of any one of clauses 1 to 31, wherein the patient is a mammal.
33. The method of any one of clauses 1 to 32, wherein the patient is a human.
34. The method of any one of clauses 1 to 33, wherein the administering is oral administration.
35. The method of clause 34, wherein the oral administration comprises administering the composition as a capsule, a tablet, a powder, a liquid suspension, a solution, an elixir, a syrup, or a concentrated drop.
36. The method of any one of clauses 1 to 33, wherein the administering is inhalation administration.
37. The method of clause 36, wherein the inhalation administration comprises administering the composition as a dry powder, a solution, a suspension, or an aerosol.
38. The method of any one of clauses 1 to 33, wherein the administering is topical administration.
39. The method of clause 38, wherein the topical administration comprises administering the composition as a lotion, a gel, a cream, an ointment, an oil, a solution, a suspension, or an emulsion.
40. The method of any one of clauses 1 to 33, wherein the administering is parenteral administration.
41. The method of any one of clauses 1 to 33, wherein the administering is administration via a needle.
42. The method of any one of clauses 1 to 33, 38, and 39, wherein the administering comprises administering as a gel comprising a polyhydric alcohol.
43. The method of any one of clauses 1 to 42, wherein the administering is carried out as a multiple dose regimen.
44. The method of clause 43, wherein the multiple dose regimen is a time period of up to about one month.
45. The method of clause 43, wherein the multiple dose regimen is a time period of up to about two months.
46. The method of clause 43, wherein the multiple dose regimen is a time period of up to about three months.
47. The method of clause 43, wherein the multiple dose regimen is a time period of up to about four months.
48. The method of any one of clauses 1 to 47, wherein the administering is carried out one time per day.
49. The method of any one of clauses 1 to 47, wherein the administering is carried out two times per day.
50. The method of any one of clauses 1 to 47, wherein the administering is carried out three times per day.
51. The method of any one of clauses 1 to 47, wherein the administering is carried out four times per day.
52. The method of any one of clauses 1 to 51, wherein the administering is carried out for at least 1 day.
53. The method of any one of clauses 1 to 52, wherein the administering is carried out for at least 7 days.
54. The method of any one of clauses 1 to 53, wherein the administering is carried out for at least 14 days.
55. The method of any one of clauses 1 to 54, wherein the administering is carried out for at least 21 days.
56. The method of any one of clauses 1 to 55, wherein the other functional groups are selected from the group consisting of an amino, a carboxyl, a hydroxy, an alkoxy, a ketone, an aldehyde, and a halogen, or combinations thereof.
57. The method of any one of clauses 1 to 33, wherein the administering is intravesical administration.
58. The method of any one of clauses 1 to 33, wherein the administering is administration via a catheter.

### EXAMPLES

Examples related to the present disclosure are described below. In most cases, alternative techniques can be used. The examples are intended to be illustrative and are not limiting or restrictive of the scope of the invention as set forth in the claims.

### Example 1

### Synthesis of Nu-8

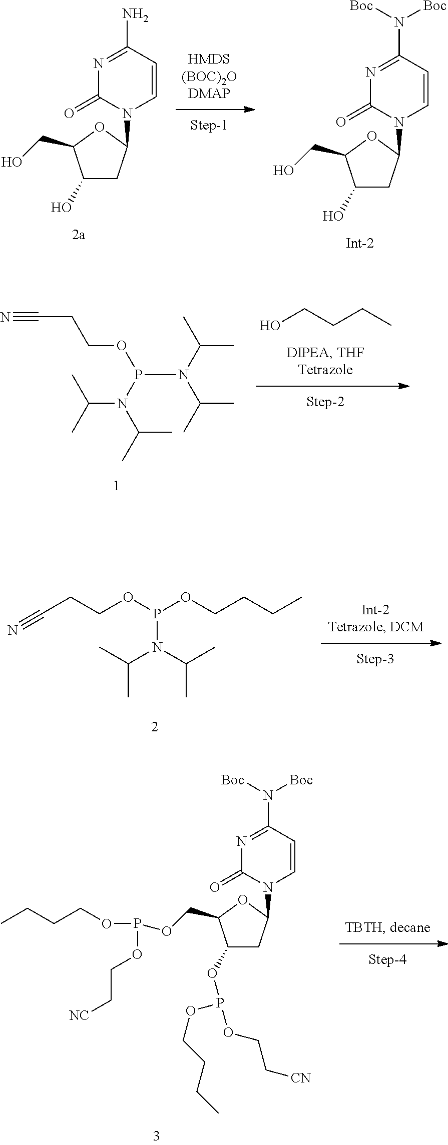

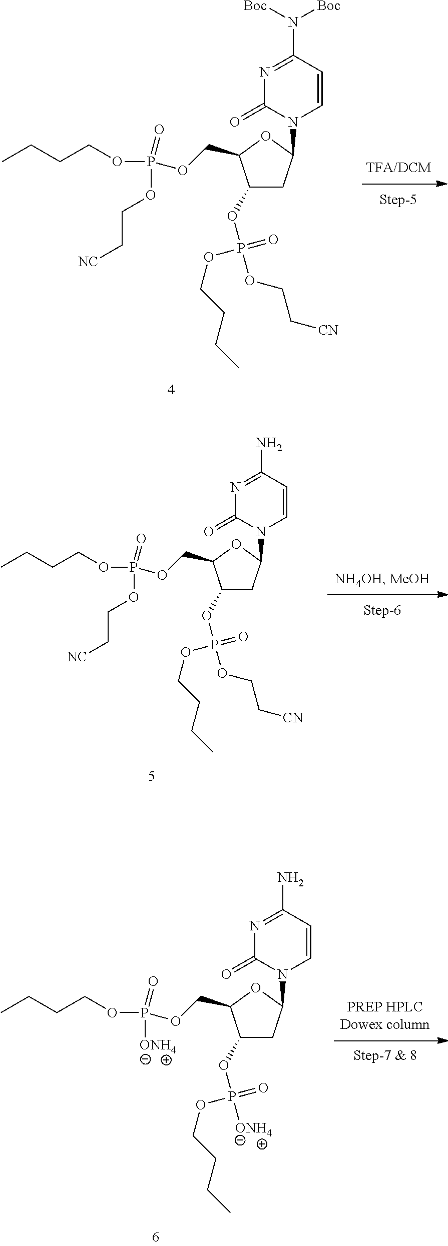

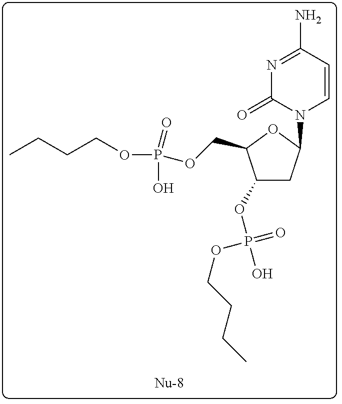 [SMILES: CCCCOP(=O)(O)OC[C@H]1O[C@@H](n2ccc(N)nc2=O)C[C@@H]1OP(=O)(O)OCCCC]

### Step-1. Protection of the Amine (Building Block Key Intermediate)

Cytidine 2a is added to hexamethyldisilazane (HMIDS), 4-(dimethylamino)pyridine (DMAP), trimethylsilyl trifluoromethanesulfonate, and di-tert-butyl dicarbonate ((BOC)₂O) in methanol to protect the nitrogen atoms of 2a by generating BOC-protected compound Int-2.

### Step-2. Preparation of Phosphitylating Reagent (Building Block Key Intermediate)

n-Butanol is reacted with the phosphinamide 1 in THE with tetrazole as a catalyst in the presence of N,N-diisopoylethylamine (DIEA). The crude product is chromatographed on neutral alumina eluting with hexane and then 2% ethyl acetate in hexane. The pure fraction is combined (by TLC) and evaporated to a residue under vacuum.

### Step-3. Coupling of Key Intermediates

BOC-protected species Int-2 is bis-phosphinylated with reagent 2 in dichloromethane (DCM)/dimethyl formamide (DMF) solvent and tetrazole as catalyst to produce 3. The reaction mixture is concentrated to a residue and the crude product is immediately oxidized in the next step.

### Steps-4 & 5. Oxidation and Amino Deprotection

The crude product 3 is oxidized with tert-butylhydroperoxide (TBTH) in the presence of decane to generate the bis-phosphonate species 4. Removal of the BOC groups is carried in DCM in the presence of trifluoroacetic acid (TFA) to yield 5. The crude product is chromatographed on silica gel eluting with ethyl acetate. The pure fractions (by TLC) are combined and evaporated to a residue under vacuum.

### Step-6. Deprotection of the Phosphodiester

Hydrolysis of 5 with methanolic ammonium hydroxide (NH₄OH, MeOH) gives crude (I) ammonium salt (VI).

### Steps-7 & 8. Purification

Purification by preparative HPLC of 6 and conversion to the free acid with Dowex 50WX8-200 resin is carried out. Evaporation of the aqueous eluate provides (I) that is diluted with purified water to provide a 20% solution at its ambient pH.

### Example 2

### Synthesis of Nu-8 Sodium Salt

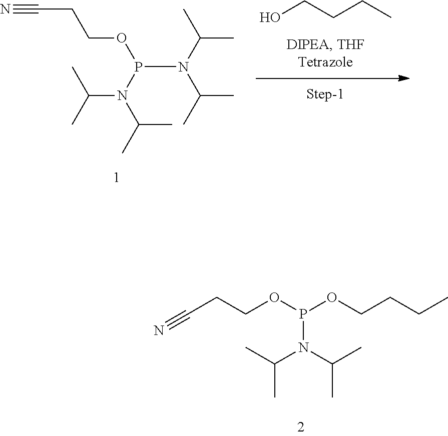

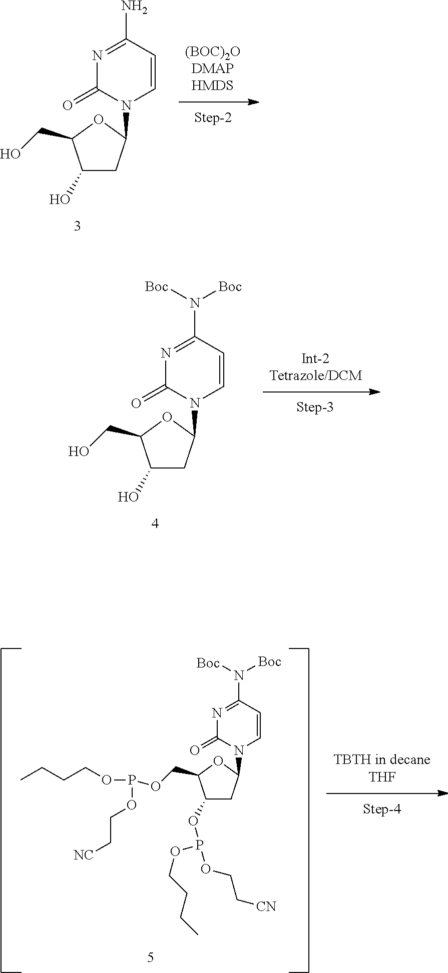

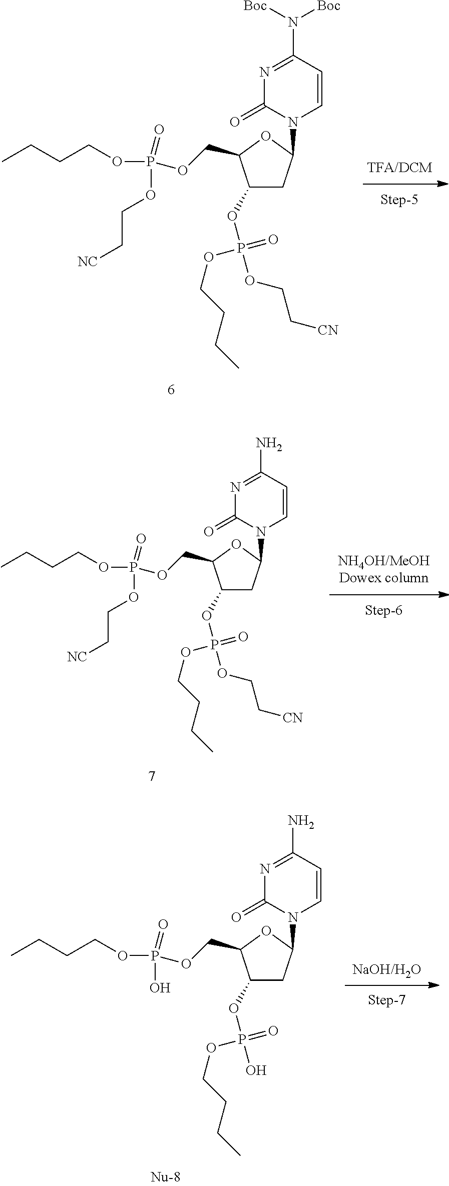 [SMILES: CCCCOP(=O)(OCCC#N)OC[C@H]1O[C@@H](n2ccc(N(C(=O)OC(C)(C)C)C(=O)OC(C)(C)C)nc2=O)C[C@@H]1OP(=O)(OCCC#N)OCCCC ; CCCCOP(=O)(OCCC#N)OC[C@H]1O[C@@H](n2ccc(N)nc2=O)C[C@@H]1OP(=O)(OCCC#N)OCCCC ; CCCCOP(=O)(O)OC[C@H]1O[C@@H](n2ccc(N)nc2=O)C[C@@H]1OP(=O)(O)OCCCC]

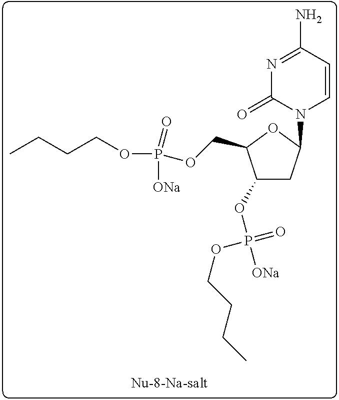

### Synthesis of Compound-2

To a solution of compound-1 (1.0 kg, 3.3222 mol) in THE (6 L) is added DIEA (1.370 mL, 8.3055 mol) and tetrazole (230 g, 3.3222 mol) followed by n-butanol (275 mL, 2.99 mol) in THF (6 L) is added drop wise at 0° C. for 12 h. The reaction mixture is stirred at room temperature for 24 h. The progress of the reaction is monitored by TLC and after completion of the reaction, solid is filtered off. Filtrate is evaporated under reduced pressure at 40° C. to afford crude compound. Crude compound is dissolved in ethyl acetate (5 L). Organic layer is washed with water (3 L) and brine (2 L). Organic layer is dried over anhydrous Na₂SO₄ filtered and the solvent is evaporated under reduced pressure to obtain crude compound. The crude compound is purified by column chromatography over basic lumina (Al₂O₃), Compound eluted with 0-2% EtOAc in petroleum ether to afford Compound-2. (700 g, 76.92%) as pale yellow liquid. H-NMR (400 MHz, chloroform-d) δ 4.18-4.07 (m, 1H), 4.02 (q, J=6.6 Hz, 1H), 3.93-3.74 (m, 4H), 2.65 (td, J=6.5, 3.6 Hz, 2H), 1.31-1.23 (m, 4H), 1.18 (dd, J=6.8, 3.8 Hz, 12H), 0.93 (td, J=7.4, 3.1 Hz, 3H). LC-MS: 275 (M+H).

### Synthesis of Compound-4

To solution of compound-3 (300 g, 1.321 mol) in hexamethyldisilazane (638 g, 3.964 mol) is added DMAP (16.11 g, 0.132 mol) followed by TMSOTf (7.22 g, 0.039 mol) is added at 0° C. and the resulting reaction mixture is stirred for 1 h at room temperature. After consumption of starting material, Boc-anhydride (1.4 L, 6.605 mol) is added at 0° C. for 1 h and the reaction mixture is stirred for 16 h at room temperature. To the reaction is added methanol (3 L) followed by triethylamine (1.5 L) is added at 0° C. for 1 h and the reaction mixture is stirred for 20 h at room temperature. Reaction mixture is concentrated under reduced pressure to get crude compound. Crude compound is diluted with ethyl acetate (3 L) and washed with water (1.0 L) and brine (1.0 L) solution; organic layer is dried over anhydrous Na₂SO₄, filtered and the solvent is evaporated under reduced pressure to get afford crude compound. The crude compound is purified by column chromatography silica gel (100-200 mesh) compound eluted 0-3% MeOH in DCM to afford Compound-4 (180 g, 31.89%) as off white solid. H-NMR (300 MHz, DMSO-d6) δ 8.41 (d, J=7.5 Hz, 1H), 6.84 (d, J=7.5 Hz, 1H), 6.06 (t, J=6.2 Hz, 1H), 5.28 (d, J=4.3 Hz, 1H), 5.07 (q, J=4.6, 4.0 Hz, 1H), 4.21 (q, J=4.1 Hz, 1H), 3.87 (q, J=3.7 Hz, 1H), 3.71-3.49 (m, 2H), 2.32 (m, 1H), 2.03 (dt, J=13.0, 6.2 Hz, 1H), 1.49 (s, 18H). LC-MS: 275 (M+H).

### Synthesis of Compound-6

To a stirred solution of compound-4 (180 g, 0.421 mol) in THE (1.0 L) is added DIEA (348 mL, 2.105 mol) and tetrazole (176 g, 2.526 mol) at 0° C. To the resulting reaction mixture is added a solution of compound-2 (519 g, 1.896 mol) in THE (800 mL) drop wise at 0° C. for 1 h. The reaction mixture is stirred at room temperature for 16 h. After completion of the reaction, tert-butyl peroxide in decane (505 mL, 5 M) is added drop wise at 0° C. and the reaction mixture is stirred for 6 h at room temperature. The reaction is monitored by TLC. After completion of the reaction, the reaction mixture is concentrated at 40° C. and diluted with ethyl acetate (3 L) and washed with water (1 L) and brine (1 L) solution. Organic layer is dried over anhydrous Na₂SO₄ filtered and the solvent is evaporated under reduced pressure to get afford crude compound (350 g crude). The crude compound is purified by column chromatography through silica gel (100-200 mesh) column eluted with 0-5% MeOH in DCM. All collected pure fractions are concentrated to afford pure Compound-6 (220 g, 64.83%) as a wine red liquid. H-NMR (300 MHz, DMSO-d6) δ 8.19 (dd, J=7.6, 1.3 Hz, 1H), 6.88 (d, J=7.5 Hz, 1H), 6.13 (t, J=10.5 Hz, 1H), 4.99 (s, 1H), 4.44 (s, 1H), 4.26-3.96 (m, 10H), 3.00-2.84 (m, 4H), 2.57-2.79 (m, 2H), 1.70-1.54 (m, 4H), 1.50 (s, 18H), 1.35 (m, 4H), 0.88 (qd, J=7.5, 2.5 Hz, 6H); LC-MS: 806 (M+H).

### Synthesis of Compound-7

To a solution of Compound-6 (220 g, 0.273 mol) in DCM (4.4 L) is added TFA (210 mL, 2.732 mol) dropwise at 0° C. The reaction mixture is stirred at room temperature for 24 h. The reaction is monitored by TLC. After completion of the reaction, solvent is evaporated under reduced pressure to afford crude compound. The crude compound is purified by column chromatography silica gel (230-400 mesh). Compound eluted with 0-10% MeOH in DCM. All collected pure fractions are concentrated to afford pure Compound-7 (170 g, 84.67%) as a pale yellow liquid. H-NMR (300 MHz, DMSO-d6) δ 7.61 (d, J=7.5 Hz, 1H), 7.27 (d, J=13.9 Hz, 2H), 6.19 (t, J=6.9 Hz, 1H), 5.74 (d, J=7.4 Hz, 1H), 4.96 (s, 1H), 4.10-3.93 (m, 11H), 2.93 (q, J=6.2 Hz, 4H), 2.29 (d, J=13.1 Hz, 2H), 1.61 (h, J=7.1 Hz, 4H), 1.35 (p, J=7.3 Hz, 4H), 0.89 (dq, J=7.9, 4.2 Hz, 6H); LC-MS: 606 (M+H).

### Synthesis of Nu-8

To a stirred solution of Compound-7 (720 g, 1.1900 mol) in MeOH (5.0 L) is added aq. ammonia (600 mL) at 0° C. The reaction mixture is stirred at room temperature for 4 h. The reaction is monitored by TLC. After completion of the reaction, evaporate the MeOH under reduced pressure the aqueous layer is washed with DCM (1.5 L). The aqueous layer is passed through Dowex-H⁺ resin. The water is removed under reduced pressure to afford Nu-8 (260 g, 43.84%) as an off white solid. H-NMR (300 MHz, DMSO-d6) δ 8.94 (s, 1H), 8.49 (s, 1H), 7.97 (d, J=7.8 Hz, 1H), 6.08 (t, J=6.1 Hz, 1H), 5.95 (d, J=7.7 Hz, 1H), 4.76 (q, J=5.8 Hz, 1H), 4.15 (q, J=4.1 Hz, 1H), 4.08 (s, 1H), 3.83 (m, 6H), 2.43 (t, J=5.6 Hz, 2H), 1.67-1.44 (m, 4H), 1.44-1.26 (m, 4H), 0.95-0.82 (m, 6H), LC-MS: 500.15 (M+H).

### Synthesis of Nu-8 Sodium Salt

To a stirred solution of compound-Nu-8 (260 g, 0.478 mol) in water (2.6 L), 1 N NaOH (950 mL) is added dropwise at 0° C. The reaction mixture is stirred at room temperature for 2 h. The reaction is monitored by TLC. After completion of the reaction, aqueous layer is washed with DCM (1.5 L). The aqueous layer is evaporated under reduced pressure to afford Nu-8 sodium salt (265 g, 93%) as off white solid. H-NMR (300 MHz, DMSO-d6) δ 7.81 (d, J=7.2 Hz, 1H), 7.2 (bs, 1H), 7.0 (bs, 1H), 6.16 (t, J=4 Hz, 1H), 5.71 (d, J=7.6 Hz, 1H), 4.69 (bs, 1H), 3.75 (m, 1H), 3.71 (m, 1H), 3.8 (m, 4H), 2.2 (q, 1H), 1.89-1.96-1.44 (m, 1H), 1.49-1.39 (m, 4H), 1.34-1.23 (m, 4H), 0.88-0.84 (m, 6H).

### Example 3

### Use of Nu-3 and Nu-8 in the Treatment of SARS-CoV-2 in Syrian Hamsters

### Animals

The hamster strain used in this study is Golden Syrian Hamsters ranging from 6 to 8 weeks in age and 74 to 110 grams in weight. Hamsters are housed three to a cage during the acclimatization period and single-housed in separate cages after exposure to virus, with free access to food and water in accordance with NIH guidelines.

### Viral Strain

The viral strain of SARS-CoV-2 used in this hamster model is SARS-CoV-2 WAS-Calu-3.

### Infection and Treatment

Syrian hamsters were infected with SARS-CoV-2 via the nose on day 1, and then treated with either Nu-3 or Nu-8 by nebulizer with aerosolized solution for 3 days (once daily for 15 minutes; volume delivered 1.5 ml 10% Nu-8, or 1.0 ml 10% Nu-3 with the first treatment administered 24 hours post-infection). One control group was not infected with SARS-CoV-2. A second control group was administered the vehicle alone. Body weights were measured daily, and lungs were harvested on day 7 and viral titer measured.

### Sampling

Following challenge, bodyweights were recorded once daily and clinical observations were recorded twice daily. CoVid-scoring was conducted during morning sessions. Oral swabs were collected at 2, 4, and 7 days post challenge and assessed for viral burden by qRT-PCR assay, by measuring both subgenomic E messenger RNA and total viral RNA. The oral cavity was swabbed with a sterile swab that was immediately placed into a cryovial with 1 mL PBS. Vials were snap-frozen on dry ice and stored at −80° C. until testing. Viral RNA was extracted from 200 μl of oral swab material using the Qiagen EZ1 DSP Virus kit on the automated EZ1 XL Advance instrument (Qiagen, Valencia, CA). Real-time quantitative reverse transcription—polymerase chain reactions (RT-qPCR) were performed on the 7500 Dx Fast thermal cycler (Thermo Fisher Scientific, Life Technologies, Carlsbad, CA). SARS-CoV-2 specific forward and reverse primers and probe targeting the E gene encoding the envelope protein were used for amplification of the viral RNA. Amplification of the sgmRNA was achieved using the Leader TRS sequence specific primer, the reverse E primer and the E specific probe as described previously 18. A synthetic RNA for subgenomic E was used as a calibrator. Final results were reported in copies/ml.

At study termination, 7 days postinfection, all animals were terminally anesthetized by ketamine/xylazine, followed by exsanguination by cardiac puncture and euthanasia. Lung tissues were collected, right lobe was sectioned (0.1-0.2 g) and snap frozen for TCID50 assay. Virus titers in lungs were determined by means of plaque assays in VeroE6 TMPRSS2 cells. Vero TMPRSS2 cells were plated at 25,000 cells per well in DMEM supplemented with 10% FBS and gentamicin. Cells were incubated at 37° C., 5.0% CO2. When cells achieved 80-100% confluency, the media was aspirated and replaced with 180 μL of DMEM containing 2% FBS and gentamicin. Frozen tissue was placed in 15 mL conical tube on wet ice containing 0.5 mL media and homogenized 10-30 secs. The tissue homogenate was spun to remove debris at 2000 g, 4° C. for 10 min. The supernatant was then passed through a strainer into the original vial and kept on wet ice. From the strained supernatant, 20 μL aliquots were tested in quadruplicate in a 96-well plate format. The top row of the 96-well plate was mixed 5 times and serially diluted by pipette transfer of 20 μL, representing 10-fold dilutions. Pipette tips were disposed of between each row and mixing was repeated until the last row on the plate. After incubation for 4 days, wells were visually inspected for cytopathic effects (CPE) scored as CPE minus (−) where non-infected wells have a clear confluent cell layer, or CPE plus (+), where rounding of infected cells is observed. Positive controls were utilized for optimal assay performance, where the TCID50 tested within 2-fold of the expected value. TCID50 of all samples were calculated using the Read-Muench formula.

### Weight Behavior of Wild Type Hamsters

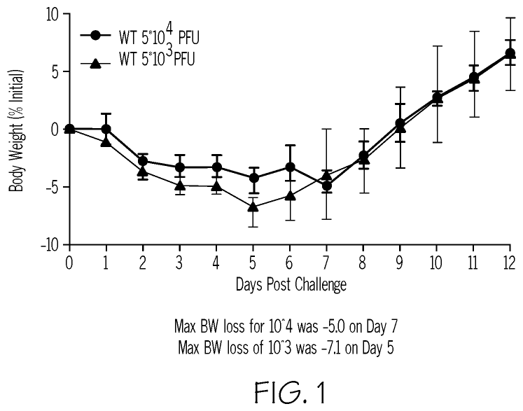

FIG. 1 provides body weight measurements for a set of hamsters infected with 5×10⁴ plaque-forming units (hereinafter “PFU”) SARS-CoV-2 and a set of hamsters infected with 5×10⁴ PFU SARS-CoV-2. Animal body weight is a good indicator of the health of the animals. A healthy hamster will gain weight steadily over time, but an infected hamster is expected to have decreased appetite for at least a portion of the time they are infected. The infected hamsters are, therefore, expected to lose weight over the course of the illness and then gradually gain weight as the infection subsides. As shown in FIG. 1, both sets of infected hamsters lost weight for the first 5 or 6 days at which point they reached a minimum weight and started gaining weight again around day 6 or 7 as they are recovering from the infection and starting to eat well again.

### Weight Behavior of Treated Hamsters

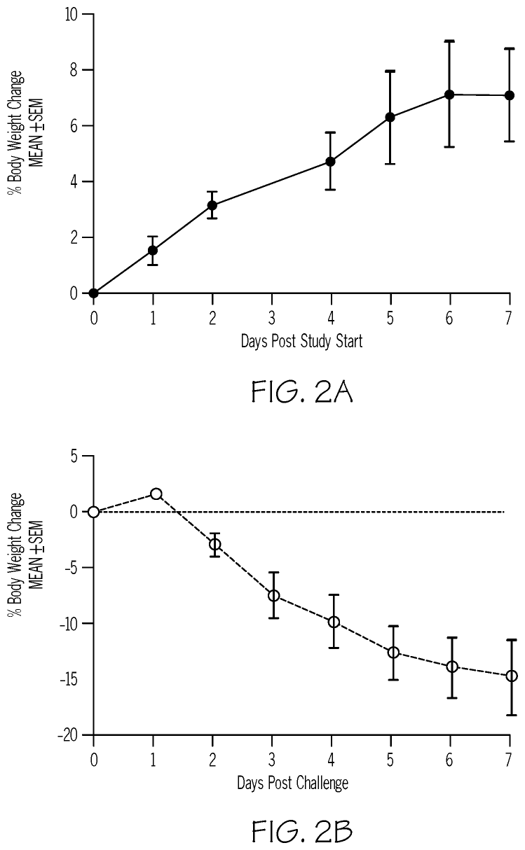

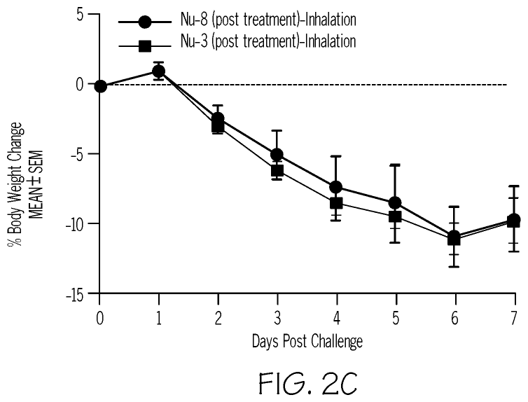

FIG. 2A provides body weight measurements of uninfected, untreated control hamsters up to seven days after the start of the study. FIG. 2B provides body weight measurements of the infected, untreated control hamsters that received only the drug vehicle. FIG. 2C provides body weight measurements of a population of hamsters treated with Nu-8 (compound of Formula (VIII)) and a population of hamsters treated with Nu-3 (compound of Formula (V)). The body weights of the uninfected, untreated control animals increase throughout the whole study as the animals are totally healthy at all times. The infected groups, including both the untreated and the two treated groups, initially mimic the body weight variation discussed above as to FIG. 1. That is, infected hamsters lost weight for the first 5 or 6 days. However, the untreated hamsters (FIG. 2B) continued to display a loss in body weight after day 6, but the treated hamsters (FIG. 2C) began to regain body weight, as shown with the body weight increase on day 7.

Viral Titers from Harvested Lungs

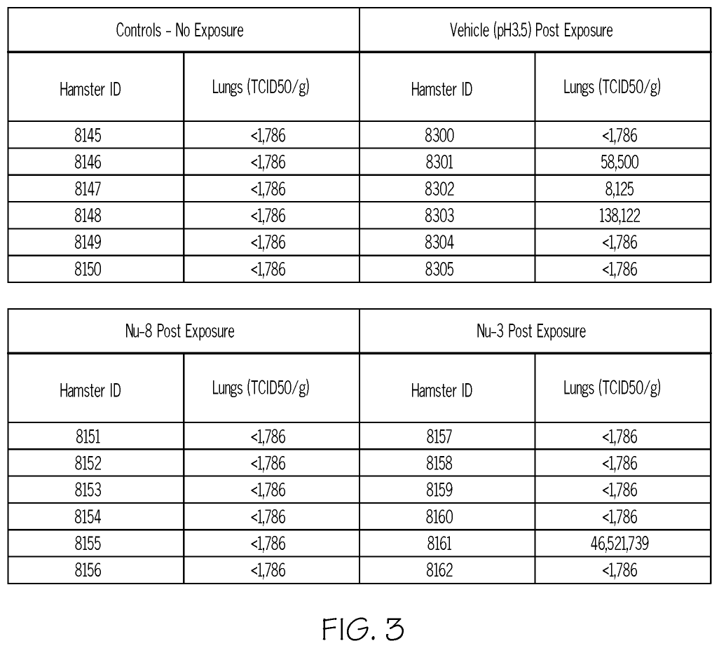

FIG. 3 shows the viral titers from the harvested lungs. The designation “<1,786” indicates that the virus was undetectable in the sample, the lowest level of detection being 1,786 median tissue culture infectious dose per gram (TCID₅₀/g). Therefore, this measurement is a total absence of viral particles. As shown in FIG. 3, in 6 out of 6 animals treated with Nu-8, there was no virus detected. Similarly, animals treated with Nu-3 also did not show any measurable viral particles, with the exception of one hamster. It is believed that experimental error led to this result.

### Example 4

### Preparation of ((2R,3S,5R)-5-(4-amino-2-oxopyrimidin-1(2H)-yl)-3-((hydroxy(isobutoxy)phosphoryl)-oxy)tetrahydrofuran-2-yl)methyl isobutyl hydrogen phosphate (Nu-13)

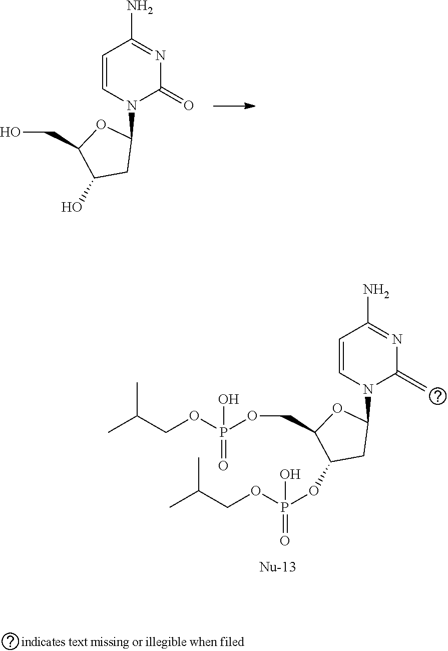 [SMILES: Nc1ccn([C@H]2C[C@H](O)[C@@H](CO)O2)c(=O)n1 ; C=C1N=C(N)C=CN1[C@H]1C[C@H](OP(=O)(O)OCC(C)C)[C@@H](COP(=O)(O)OCC(C)C)O1]

To a stirred solution of 4-amino-1-((2R,4S,5R)-4-hydroxy-5-(hydroxymethyl) tetrahydrofuran-2-yl)pyrimidin-2(1H)-one (Cas No 951-77-9) (2.5 g, 11.00 mmol) in trimethyl 0 phosphate (25 ml) with 4 Å molecular sieve (w/w) was added phosphorous oxychloride (2.5 ml, 26.40 mmol) at 0° C. under a N₂ atmosphere and stirred at 0° C. for 2 h. Then isobutanol (3 ml, 33.00 mmol) was added to the reaction mixture and the reaction was stirred at 0° C. for another 2 h. The reaction was monitored by LCMS and after completion the reaction mixture was poured into diethyl ether (500 ml) and quenched with 10% saturated sodium bicarbonate solution (30 ml). The aqueous layer was acidified by 1N HCl (15 ml) to pH˜2.0 and lyophilized to obtain the crude compound which was purified by preparative HPLC to afford Nu-13 (501.96 mg, 9% yield) as a white solid.

Preparative HPLC Conditions: Column/dimensions: SUN FIRE C18(19*150 mm), 5μ; mobile phase A: 0.1% TFA in water; mobile phase B: CH₃CN; flow rate: 18 ml/min; gradient (Time/% B): 0/5, 1/5, 12/30, 12.1/100, 16/100, 16.1/5, 18/5; solubility: CH₃CN+THF; LCMS: 91%; 500.48 [M+H]⁺; UPLC: 92%; ¹H NMR (400 MHz, DMSO-d6): δ 8.02 (s, 1H), 7.82 (d, J=7.2 Hz, 1H), 7.57 (s, 1H), 6.08 (t, J=6 Hz, 1H), 5.81 (d, J=7.6 Hz, 1H), 4.77 (s, 1H), 4.10-4.00 (m, 3H), 3.78-3.64 (m, 1H), 3.63-3.49 (m, 4H), 2.39-2.32 (m, 1H), 2.27-2.25 (m, 1H), 1.85-1.77 (m, 2H), 0.91-0.84 (m, 12H).

### Example 5

### Preparation of ((2R,3S,5R)-5-(4-amino-2-oxopyrimidin-1(2H)-yl)-3-(((benzyloxy)(hydroxy)-phosphoryl)oxy)tetrahydrofuran-2-yl)methyl benzyl hydrogen phosphate (Nu-14)

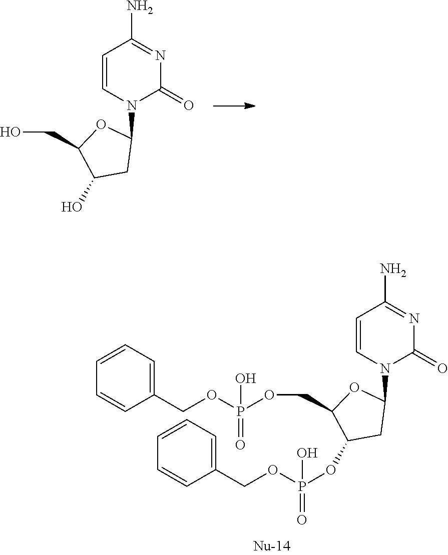 [SMILES: Nc1ccn([C@H]2C[C@H](O)[C@@H](CO)O2)c(=O)n1 ; Nc1ccn([C@H]2C[C@H](OP(=O)(O)OCc3ccccc3)[C@@H](COP(=O)(O)OCc3ccccc3)O2)c(=O)n1]

To a stirred solution of 4-amino-1-((2R,4S,5R)-4-hydroxy-5-(hydroxymethyl) tetrahydrofuran-2-yl)pyrimidin-2(1H)-one (Cas No 951-77-9) (2.5 g, 11.00 mmol) in trimethyl 0 phosphate (25 ml) with 4 Å molecular sieve (w/w) was added phosphorous oxychloride (5.14 ml, 55.01 mmol) at 0° C. under a N₂ atmosphere and stirred 0° C. for 2 h. Then benzyl alcohol (5.7 ml, 55.01 mmol) was added, and reaction mixture was stirred at 0° C. for another 2 h. The reaction was monitored by LCMS and after completion the reaction mixture was poured in diethyl ether (500 ml) and quenched with 10% saturated sodium bicarbonate solution (30 ml). The aqueous layer was acidified with 1N HCl (15 ml) to pH˜2.0 and lyophilized to obtain the crude compound which was purified by preparative HPLC to afford Nu-14 (550 mg, 7.2% yield) as a white solid.

Preparative HPLC conditions: Column/dimensions: Luna omega (19*250, Sum); mobile phase A: 0.1% TFA in water; mobile phase B: CH₃CN; gradient (Time/% B): 0/5, 2/5, 6/20, 16/20, 16.1/100, 20/100, 20.1/5, 23/5; flow rate: 18 ml/min; solubility: THF; LCMS: 95%; 568.33 [M+H]⁺; UPLC: 93%; ¹H NMR 400 MHz, DMSO-d6: δ 8.49 (bs, 1H), 7.91-7.84 (m, 2H), 7.37-7.27 (m, 10H), 6.06 (t, J=6.4 Hz, 1H), 5.87 (d, J=7.6 Hz, 1H), 4.93-4.80 (m, 5H), 4.17-3.90 (m, 3H), 2.38-2.32 (m, 1H), 2.29-2.26 (m, 1H).

### Example 6

### Preparation of ((2R,3R,4R,5R)-5-(4-amino-2-oxopyrimidin-1(2H)-yl)-3-((butoxy(hydroxy)phosphoryl)oxy)-4-methoxytetrahydrofuran-2-yl)methyl butyl hydrogen phosphate (Nu-10)

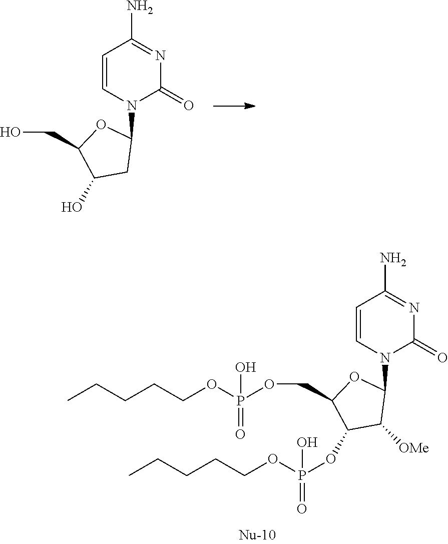 [SMILES: Nc1ccn([C@H]2C[C@H](O)[C@@H](CO)O2)c(=O)n1 ; CCCCCOP(=O)(O)OC[C@H]1O[C@@H](n2ccc(N)nc2=O)[C@H](OC)[C@@H]1OP(=O)(O)OCCCCC]

To a stirred solution of 4-amino-1-((2R,3R,4R,5R)-4-hydroxy-5-(hydroxymethyl)-3-methoxytetrahydrofuran-2-yl)pyrimidin-2(1H)-one (Cas No: 2140-72-9) (3 g, 11.66 mmol) in 0 trimethyl phosphate (30 ml) with 4 Å molecular sieve (w/w) was added phosphorous oxychloride (5.5 ml, 58.32 mmole) at 0° C. under a N₂ atmosphere and stirred at 0° C. for 2 h. Then 1-butanol (5.33 ml, 58.32 mmol) was added to the reaction mixture and was stirred at 0° C. for another 2 h. The reaction was monitored by LCMS and after completion the reaction mixture was poured into diethyl ether (600 ml) and quenched with 10% saturated sodium bicarbonate solution (36 ml). The aqueous layer was acidified with 1N HCl (18 ml) to pH˜2.0 and lyophilized to obtain crude compound which was purified by preparative HPLC to afford Nu-10 (530 mg, 6% yield) as a white solid.

Preparative HPLC Conditions: Column/dimensions: Luna omega (19*250, Sum); mobile phase A: 0.1% TFA in water; mobile phase B: CH₃CN; gradient (Time/% B): 0/10, 1/10, 5/25, 12/25, 12.1/100, 14/100, 14.1/10, 17/10; flow rate: 18 ml/min; solubility: THF; LCMS: 99%; 530.37 [M+H]⁺; UPLC: 98%; ¹H NMR (400 MHz, DMSO-d6): δ 9.00 (bs, 1H), 8.08 (d, J=8 Hz, 2H), 5.94 (d, J=7.6 Hz, 1H), 5.76 (m, 1H), 4.56-4.53 (m, 1H), 4.17-4.12 (m, 3H), 3.97-3.96 (m, 1H), 3.85-3.46 (m, 4H), 3.41 (s, 3H), 1.58-1.49 (m, 4H), 1.36-1.28 (m, 4H), 0.90-0.85 (m, 6H).

### Example 7

### Use of Nu-10 in the Treatment of SARS-CoV-2 Viral Replication in HEK293T ACE2 Cells

### Cell Line and Virus Materials

HEK293T cells expressing human ACE2 (HEK293T ACE2) (NR-52511, BEI Resources, NIAID, NIH) were cultured in complete media prepared using Dulbecco's modified Eagle medium (12100-038, Gibco) supplemented with 10% HI-FBS (16140-071, Gibco), 100 U/mL Penicillin-Streptomycin (15140122, Gibco) and GlutaMAX™ (35050-061, Gibco).

SARS-CoV-2 isolate Hong Kong/VM20001061/2020 (BEI resources NR-52282) was propagated and titrated in VeroE6 cells.

### Methods

HEK293T ACE2 cells were seeded in 24-well plates coated with poly 1 lysine (Sigma, P9155). SARS-CoV-2 infection was done at 0.01 mmol in 100 μL per well inoculum volume. The plate was rocked every 10 min to ensure even adsorption of virus to cells. The media in each well was then topped up with 400 μL infection media (containing varying concentrations of Nu-10 as per Table 1) and incubated at 37° C./5% CO₂. 48 hours post incubation, total RNA was extracted using TRIzol (Thermo Fisher, 15596018) as per manufacturer's instructions, and viral copy number was estimated by qRT PCR.

qRT-PCR: Equal amount of RNA was used to determine viral load using the AgPath-ID™ One-Step RT-PCR kit (Applied Biosystems, AM1005). The following primers and probes targeting the SARS-CoV-2 N-1 gene were used for amplification. Forward primer: 5′GACCCCAAAATCAGCGAAAT3′ (SEQ ID NO.: 1) and Reverse primer: 5′ TCTGGTTACTGCCAGTTGAATCTG3′, (SEQ ID NO.: 2) Probe: (6-FAM/BHQ-1) ACCCCGCATTACGTTTGGTGGACC (SEQ ID NO.: 3). The Ct values were used to determine viral copy numbers by generating a standard curve using the SARS-CoV-2 genomic RNA standard.

### Results

Table 1 summarizes the results from the qRT tests of the different concentrations of Nu-10 under the two sets of conditions—A: preincubation of virus with Nu-10 for 30 minutes before addition to the HEK239 ACE2 cells at two different concentrations; and B: no preincubation—Nu-10 at two different concentrations was added at the same time as the virus to the HEK293 ACE2 cells and incubated.

TABLE 1
Results of the qRT experiments

| | Nu-10 | Log10 Viral | | |
|---|---|---|---|---|
| | concentration | RNA copy | % of | % inhi- |
| | (mg/ml) | number | control | bition |
| A: Pre-incubation of | 0 | 6.6 | 100 | 0 |
| virus with Nu-10 | 3.12 | 5.15 | 8 | 92 |
| | 6.25 | 5.25 | 10 | 90 |
| B: No pre-incubation | 0 | 6.8 | 100 | 0 |
| with Nu-10 | 3.12 | 4.8 | 3 | 97 |
| | 6.25 | 6.3 | 47 | 53 |

### Example 8

### Preparation of ((2R,3S,5R)-5-(6-amino-9H-purin-9-yl)-3-(((cyclohexyloxy)(hydroxy)phosphoryl)oxy)tetrahydrofuran-2-yl)methyl cyclohexyl hydrogen phosphate (Nu-12)

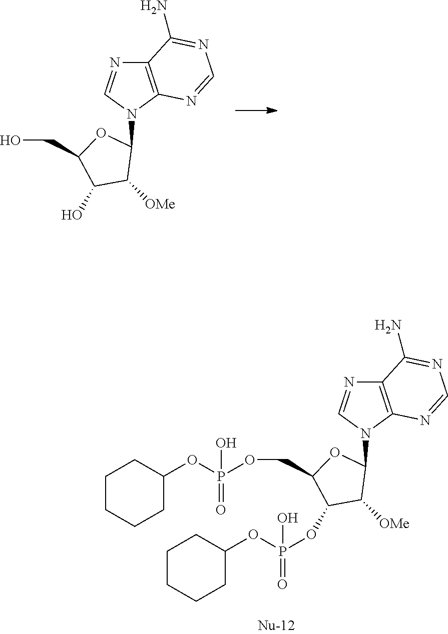 [SMILES: CO[C@@H]1[C@H](O)[C@@H](CO)O[C@H]1n1cnc2c(N)ncnc21 ; CO[C@@H]1[C@H](OP(=O)(O)OC2CCCCC2)[C@@H](COP(=O)(O)OC2CCCCC2)O[C@H]1n1cnc2c(N)ncnc21]

To a stirred solution of (2R,3S,5R)-5-(6-amino-9H-purin-9-yl)-2-(hydroxymethyl) tetrahydrofuran-3-ol (Cas No 961-07-9) (3.5 g, 13.93 mmol) in trimethyl phosphate (35 ml) with 4 Å molecular sieve (w/w) was added phosphorous oxychloride (6.5 ml, 69.65 mmol) at 0° C. under a N₂ atmosphere and stirred at 0° C. for 2 h. Then cyclohexanol (6.4 ml, 69.65 mmol) was added and reaction mixture was stirred at 0° C. for another 2 h. The reaction was monitored by LCMS and after completion the reaction mixture was poured into diethyl ether (700 ml) and quenched with 10% saturated sodium bicarbonate solution (42 ml). The aqueous layer was acidified with 1N HCl (21 ml) at pH˜2.0 and lyophilized to obtain the crude compound which was purified by preparative HPLC to afford Nu-12 (325 mg, 4%) as a white solid.

Preparative HPLC Conditions: Column/dimensions: GEMINI NX C18 (21.5*250 mm 5 μm); mobile phase A: 0.1% TFA in water (aq); mobile phase B: Acetonitrile (org); gradient (Time/% B): 0/10, 1/10, 10/45, 11.49/45, 11.50/100, 13/100, 13.10/10, 14.50/10; flow rate: 18 ml/min; solubility: Acetonitrile+THF; LCMS: 99%; 576.39 [M+H]⁺; UPLC: 96%; ¹H NMR (400 MHz, DMSO-d6): δ 8.36 (s, 1H), 8.19 (s, 1H), 7.60 (bs, 2H), 6.39 (t, J=7.2 Hz, 1H), 4.27-4.26 (m, 1H), 4.20-4.19 (m, 1H), 4.13-4.12 (m, 2H), 4.04-4.00 (m, 1H), 3.00-2.97 (m, 1H), 2.51-5.49 (m, 1H), 1.86-1.78 (m, 4H), 1.68-1.65 (m, 4H), 1.17-1.35 (m, 12H).

### Example 9

### Preparation of ((2R,3R,4R,5R)-5-(6-amino-9H-purin-9-yl)-3-((butoxy(hydroxy)phosphoryl)oxy)-4-methoxytetrahydrofuran-2-yl)methyl butyl hydrogen phosphate (Nu-11)

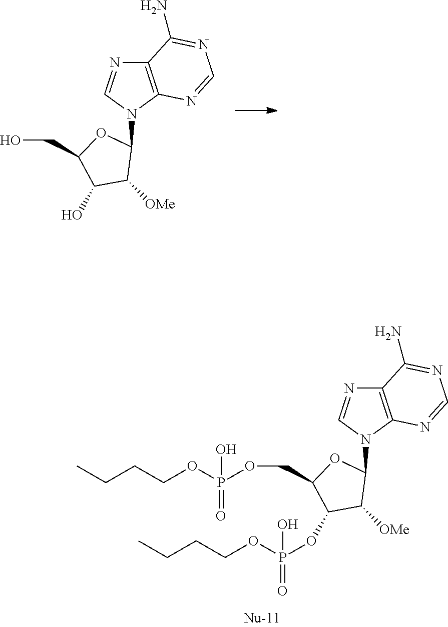 [SMILES: CO[C@@H]1[C@H](O)[C@@H](CO)O[C@H]1n1cnc2c(N)ncnc21 ; CCCCOP(=O)(O)OC[C@H]1O[C@@H](n2cnc3c(N)ncnc32)[C@H](OC)[C@@H]1OP(=O)(O)OCCCC]

To a stirred solution of (2R,3R,4R,5R)-5-(6-amino-9H-purin-9-yl)-2-(hydroxymethyl)-4-methoxytetrahydrofuran-3-ol (Cas No 2140-79-6) (5.0 g, 17.78 mmol) in trimethyl phosphate (50 ml) with 4 Å molecular sieve (w/w) was added phosphorous oxychloride (8.4 ml, 88.88 mmol) at 0° C. under a N₂ atmosphere and stirred at 0° C. for 2 h. Then n-butanol (8.1 ml, 88.88 mmol) was added, and reaction mixture was stirred at 0° C. for another 2 h. The reaction was monitored by LCMS and after completion the reaction mixture was poured in diethyl ether (1000 ml) and quenched with 10% saturated sodium bicarbonate solution (75 ml). The aqueous layer was acidified with 1N HCl (40 ml) at pH˜2.0 and lyophilized to obtain crude compound which was purified by reverse phase purification (REVELERIS RP FLASH CARTRIDGE 120G; 20% CH₃CN in 0.1% FA in water) to afford Nu-11 (1.6 g, 16% yield) as a white solid.

LCMS: 91%; 554.38 [M+H]⁺; UPLC: 92%; ¹H NMR (400 MHz, DMSO-d6): δ 8.40 (s, 1H), 8.21 (s, 1H), 7.68 (bs, 2H), 6.05 (d, 1H), 5.09-4.98 (m, 1H), 4.61-4.32 (m, 1H), 4.35-4.36 (m, 1H), 4.20-4.05 (m, 2H), 3.93-3.80 (m, 4H), 3.38 (s, 3H), 1.58-1.48 (m, 4H), 1.36-1.27 (m, 4H), 0.90-0.82 (m, 6H).

### Example 10

### Use of Nu-10 and Nu-11 to Neutralize SARS-CoV-2 Infection In Vitro

Vero cells, derived from the kidney of an African green monkey, were utilized to evaluate the antiviral activity of test items against Pseudovirus rVSV-SARS-CoV-2. Vesicular stomatitis virus (VSV) lacking glycoprotein (G) has been pseudotyped with the wild-type (Wuhan) glycoprotein (G) of SARS-CoV-2. This system contains a luciferase reporter gene which was used to assay readout.

Vero cells were seeded in growth medium (Eagle's Minimum Essential Medium) in black 96-well plates at 5×10⁴ cells per well and incubated overnight (16-20 hours) at 37° C. with 5% CO2. Serial dilutions of the test articles from stock solutions were prepared in Vero infection medium to twice the final concentration. Pseudovirus rVSV-SARS-CoV-2 was diluted in Vero infection medium and was added to the diluted test articles in a 1:1 ratio.

Vero growth medium was aspirated from the cells and 100 μL of the mixture of diluted test articles and pseudovirus was added to the cells in triplicate. Cells were incubated at 37° C. with 5% CO2 for 24 hours. After 24 hours of infection, 100 μL of Bright Glo™ Luciferase Assay System was added directly to the cells without removing the inoculum. The plate was incubated at room temperature for 10 minutes to stabilize the luminescence. The luminescence of each well was then read by a plate reader.

Luminescence data in relative light units (RLU) was imported into Excel. Data was normalized to the average absorbance of the virus-only wells, considered as 0% inhibition, to calculate % inhibition values. The % inhibition values were graphed using a sigmoidal dose-response model and NT50 values were derived from the inhibition curves.

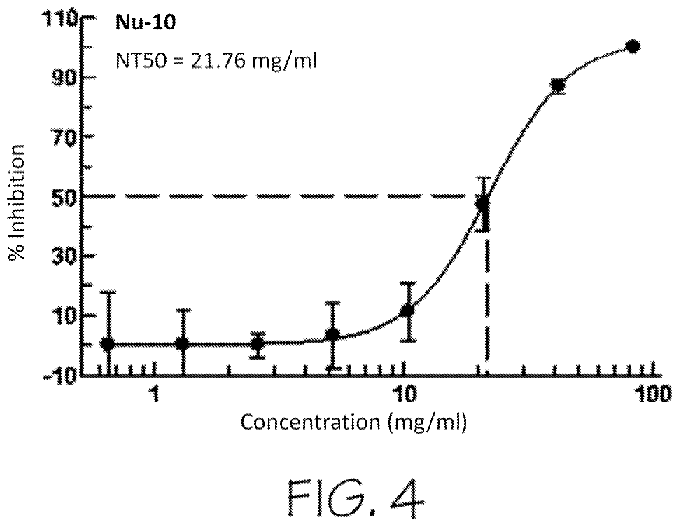

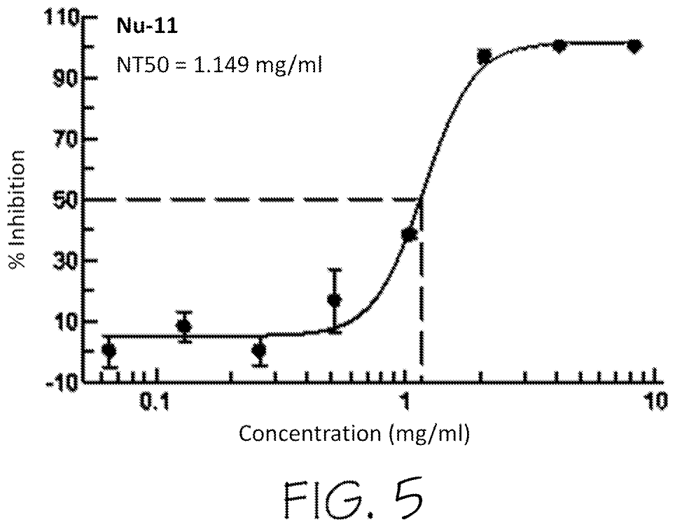

FIGS. 4 and 5 show that Bisphosphocins, i.e., Nu-10 and Nu-11, respectively, neutralize SARS CoV-2 infection in vitro. As noted above, Vero cells were infected with Pseudovirus rVSV-SARS-CoV-2 in the presence of test items. After 24 hours of post-infection, the expression of the reporter gene (luciferase) was quantified by measuring the luminescence of each well. Data shown for test items represents the average and standard deviation of triplicates.

Bisphosphocins Nu-10 and Nu-11 both neutralized SARS-CoV-2 infections in in vitro assays as revealed with the immunostaining procedure. Nu-11 provided the better NT50 value (1.149 mg/ml vs. 21.76 mg/ml) in Pseudoviral neutralization assays. These results indicate that Bisphosphocins were effective in abrogating SARS-CoV-2 infections in in vitro assays and the observed antiviral effects were likely due to the direct effects of the drug on virus particles.

While embodiments have been disclosed hereinabove, the present invention is not limited to the disclosed embodiments. Instead, this application is intended to cover any variations, uses, or adaptations of the invention using its general principles. Further, this application is intended to cover such departures from the present disclosure as come within known or customary practice in the art to which this invention pertains and which fall within the limits of the appended claims.

1. A method for treating a coronavirus infection in a patient in need thereof, the method comprising:
administering an effective amount of a composition comprising a compound having the formula): 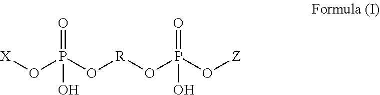
wherein:
Z and X are independently selected from alkyl, alkyloxyalkyl, alkylaryl, aryl, alkenyl, arylalkyl, arylalkenyl, or alkylarylalkenyl, and each substituent, Z or X, is optionally substituted with other functional groups; and
R is selected from alkyl, monocyclic alkyl, bicyclic alkyl, polycyclic alkyl, monocyclic heterocycle, bicyclic heterocycle, polycyclic heterocycle, alkyloxyalkyl, alkylaminoalkyl, alkylaryl, aryl, heteroaryl, alkenyl, arylalkyl, arylalkenyl, or alkylarylalkenyl, and each substituent, R, is optionally substituted with other functional groups; or
a pharmaceutically acceptable salt thereof.

2. The method of claim 1, wherein the coronavirus is selected from the group consisting of an alphacoronavirus, a betacoronavirus, a gammacoronavirus, a deltacoronavirus, and an omicroncoronavirus, or combinations thereof.

3. The method of claim 1, wherein the coronavirus is selected from the group consisting of porcine epidemic diarrhea virus (PEDv), scotophilus bat coronavirus 512, bat coronavirus CDPHE15, BtRF-Alpha-CoV HuB-2013, bat coronavirus HKU10, miniopterus bat coronavirus HKU8, miniopterus bat coronavirus 1, Nyctalus velutinus alphacoronavirus SC-2013, Pipistrellus kuhli coronavirus 3398, Myotis ricketti alphacoronavirus Sax-2011, HumCoV 229E, 229E-related bat coronavirus, camel alphacoronavirus, alpaca respiratory coronavirus, HumCoV NL63, NL63-related Bat-CoV BtKYNL63-9b, HKU2, SADSr-CoV, SADS-CoV, Lucheng Rn rat coronavirus2, FIPV, TGEV, PRCV, alphacoronavirus 1, mink coronavirus 1, FRCoV-NL-2010, Sorex araneus coronavirus T14, Suncus murinus coronavirus X74, HumCoV OC43, HumCoV OC43 isolate TNP F1778 2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, bat Hp-betacoronavirus Zhejiang2013, peafowl IBV, avian coronavirus 9203, fowl IBV, avian coronavirus, duck-CoV, partridge IBV, goose coronavirus CB17, beluga whale coronavirus SW1, HKU22, HKU19, HKU20, HKU21, HKU11, HKU12, HKU16, HKU27, HKU28, HKU29, HKU18, HKU13, HKU30-UAE, HKU30-Poland, HKU17-USA, HKU17-China, AlCCoV, and HKU15, or combinations thereof.

4. The method of claim 1, wherein the coronavirus is a betacoronavirus selected from the group consisting of HumCoV OC43 isolate TNP F1778_2, HKU23, BovCoV, SACoV, GiCoV, bovine coronavirus isolate alpaca, canine respiratory coronavirus K37, PHEV, equine coronavirus, HKU14, HKU24, myodes coronavirus 2JL14, HKU1, MHV, rat coronavirus Parker, HKU4, HKU5, human MERS-CoV, camel MERS-CoV, hedgehog coronavirus 1, HKU9, rousettus bat coronavirus GCCDC1, eidolon bat coronavirus C704, human SARS-CoV, palm civet SARS-CoV, badger SARS-CoV, bat-SL-CoV RsSHC014, bat-SL-CoV Rs3367, bat-SL-CoV WIV1, HKU3, bat-SL-CoV ZC45, bat-SL-CoV ZXC21, bat-CoV RaTG13, bat-CoV RmYN02, human SARS-CoV-2, cat-CoV, tiger-CoV, dog-CoV, mink-CoV, GD pangolin-CoV, and bat Hp-betacoronavirus Zhejiang2013, or combinations thereof.

5. The method of claim 1, wherein the coronavirus is human SARS-CoV-2.

6. The method of claim 1, wherein the coronavirus is human SARS-CoV.

7. The method of claim 1, wherein the compound comprises a compound having the formula: 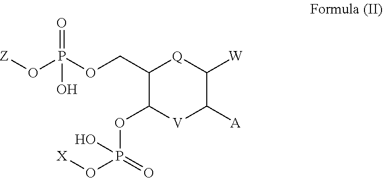
wherein:
Z and X are independently selected from alkyl, alkylaryl, aryl, alkenyl, arylalkyl, arylalkenyl, or alkylarylalkenyl, and each substituent, Z or X, is optionally substituted with other functional groups;
Q is O or S;
V is a bond, O, or S;
A is H, alkyl, or alkyloxy, and A is optionally substituted with other functional groups; and
W is a monocyclic or bicyclic heterocycle; or
a pharmaceutically acceptable salt thereof.

8. The method of claim 7, wherein the bond to W is to a heteroatom of the heterocycle.

9. The method of claim 7, wherein the bond to W is to a carbon atom of the heterocycle.

10. The method of claim 1, wherein the compound comprises a compound having the formula: 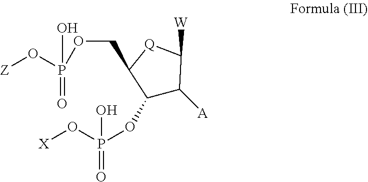
wherein:
Z and X are independently selected from alkyl, alkylaryl, aryl, alkenyl, arylalkyl, arylalkenyl, or alkylarylalkenyl, and each substituent, Z or X, is optionally substituted with other functional groups;
Q is O or S;
A is H, alkyl, or alkyloxy, and A is optionally substituted with other functional groups; and
W is a monocyclic or bicyclic heterocycle; or
a pharmaceutically acceptable salt thereof.

11. The method of claim 10, wherein the bond to W is to a heteroatom of the heterocycle.

12. The method of claim 10, wherein the bond to W is to a carbon atom of the heterocycle.

13. The method of claim 1, wherein Z and X are independently alkyl and are substituted with at least one hydroxyl group.

14. The method of claim 1, wherein Z and X are both 4-hydroxybutyl.

15. The method of claim 1, wherein Z and X are both n-butyl.

16. The method of claim 1, wherein the compound is selected from the group consisting of a compound having the formula: 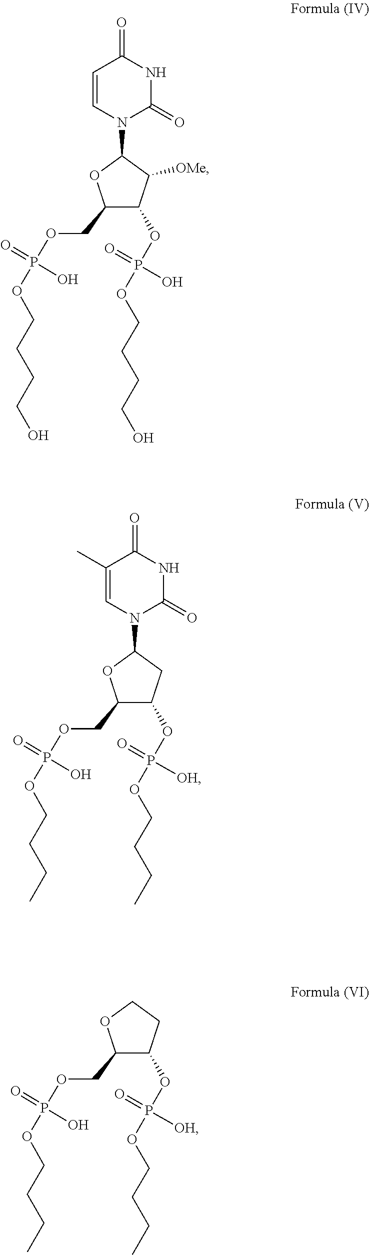 [SMILES: CO[C@@H]1[C@H](OP(=O)(O)OCCCCO)[C@@H](COP(=O)(O)OCCCCO)O[C@H]1n1ccc(=O)[nH]c1=O ; CCCCOP(=O)(O)OC[C@H]1O[C@@H](n2cc(C)c(=O)[nH]c2=O)C[C@@H]1OP(=O)(O)OCCCC ; CCCCOP(=O)(O)OC[C@H]1OCC[C@@H]1OP(=O)(O)OCCCC] 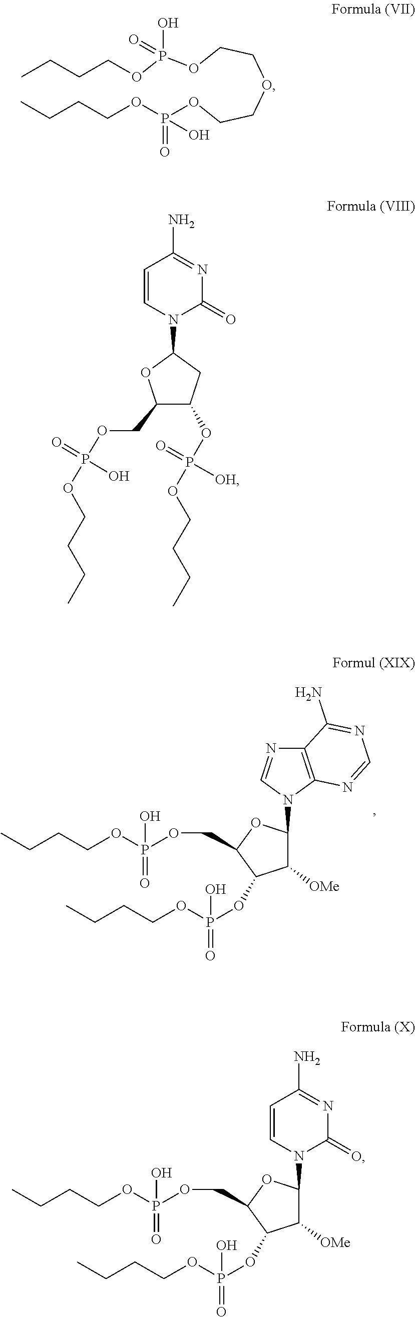 [SMILES: CCCCOP(=O)(O)OCCOCCOP(=O)(O)OCCCC ; CCCCOP(=O)(O)OC[C@H]1O[C@@H](n2ccc(N)nc2=O)C[C@@H]1OP(=O)(O)OCCCC ; CCCCOP(=O)(O)OC[C@H]1O[C@@H](n2cnc3c(N)ncnc32)[C@H](OC)[C@@H]1OP(=O)(O)OCCCC ; CCCCOP(=O)(O)OC[C@H]1O[C@@H](n2ccc(N)nc2=O)[C@H](OC)[C@@H]1OP(=O)(O)OCCCC] 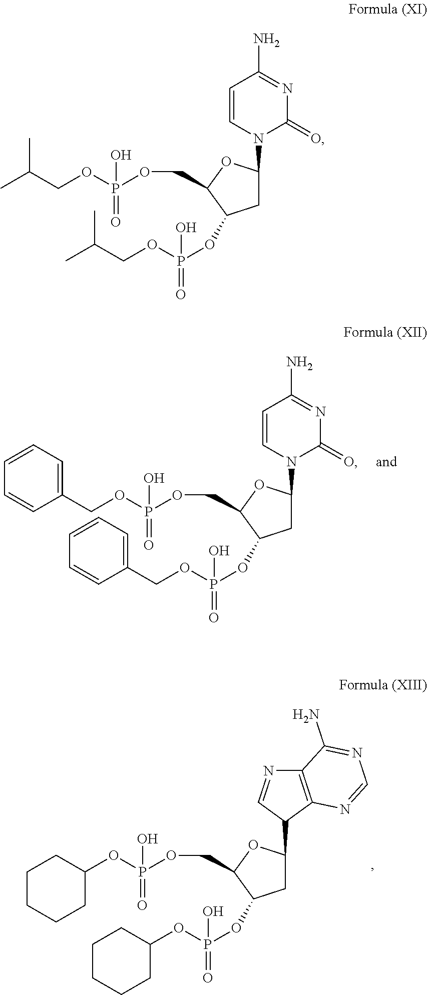 [SMILES: CC(C)COP(=O)(O)OC[C@H]1O[C@@H](n2ccc(N)nc2=O)C[C@@H]1OP(=O)(O)OCC(C)C ; Nc1ccn([C@H]2C[C@H](OP(=O)(O)OCc3ccccc3)[C@@H](COP(=O)(O)OCc3ccccc3)O2)c(=O)n1 ; "and" ; Nc1ncnc2c1N=CC2[C@H]1C[C@H](OP(=O)(O)OC2CCCCC2)[C@@H](COP(=O)(O)OC2CCCCC2)O1]
or combinations thereof; or
a pharmaceutically acceptable salt thereof.

17. (canceled)

18. (canceled)

19. (canceled)

20. (canceled)

21. (canceled)

22. (canceled)

23. (canceled)

24. (canceled)

25. (canceled)

26. (canceled)

27. The method of claim 1, wherein the pharmaceutically acceptable salt is selected from the group consisting of an ammonium salt, a calcium salt, a sodium salt, a potassium salt, a magnesium salt, and a cobalt salt, or combinations thereof.

28. (canceled)

29. (canceled)

30. The method of claim 1, wherein the composition further comprises one or more pharmaceutically acceptable carriers, excipients, or diluents.

31. The method of claim 1, wherein the composition further comprises at least one additional active ingredient.

32. (canceled)

33. The method of claim 1, wherein the patient is a human.

34. The method of claim 1, wherein the administering is selected from an oral administration, an inhalation administration, a topical administration, a parenteral administration, and/or an administration via a needle.

35. (canceled)

36. (canceled)

37. (canceled)

38. (canceled)

39. (canceled)

40. (canceled)

41. (canceled)

42. (canceled)

43. The method of claim 1, wherein the administering is carried out as a multiple dose regimen.

44. (canceled)

45. (canceled)

46. (canceled)

47. (canceled)

48. (canceled)

49. (canceled)

50. (canceled)

51. The method of claim 1, wherein the administering is carried out up to four times per day.

52. The method of claim 1, wherein the administering is carried out for at least 1 day.

53. (canceled)

54. (canceled)

55. (canceled)

56. The method of claim 1, wherein the other functional groups are selected from the group consisting of an amino, a carboxyl, a hydroxy, an alkoxy, a ketone, an aldehyde, and a halogen, or combinations thereof.


SEQUENCE LISTING (3 sequences)

SEQ ID NO: 1 - DNA, 20 bp, synthetic construct
  source 1..20: mol_type = other DNA
gaccccaaaatcagcgaaat

SEQ ID NO: 2 - DNA, 24 bp, synthetic construct
  source 1..24: mol_type = other DNA
tctggttactgccagttgaatctg

SEQ ID NO: 3 - DNA, 24 bp, synthetic construct
  source 1..24: mol_type = other DNA
accccgcattacgtttggtggacc